SEQUENCE LISTING (30 sequences)

SEQ ID NO: 1 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGVRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 2 - DNA, 1707 bp, Zymomonas mobilis
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctag

SEQ ID NO: 3 - DNA, 1707 bp, Artificial Sequence
  Encoding SEQ ID NO 1, codon-optimized for Lactobacillus paracasei
atgtcatataccgttggcacctatttggctgaacgtttggttcaaatcggcttgaagcaccacttcgctgttgctggcgattataacttggttttgttggataacttgttgttgaacaagaacatggaacaagtttattgctgcaacgaattgaactgcggcttctcagctgaaggctatgctcgtgctaagggcgctgctgctgctgttgttacctattcagttggcgctttgtcagctttcgatgctatcggcggcgcttatgctgaaaacttgccagttatcttgatctcaggcgctccaaacaacaacgatcacgctgctggccacgttttgcaccacgctttgggcaagaccgattatcactatcaattggaaatggctaagaacatcaccgctgctgctgaagctatctataccccagaagaagctccagctaagatcgatcacgttatcaagaccgctttgcgtgaaaagaagccagtttatttggaaatcgcttgcaacatcgcttcaatgccatgcgctgctccaggcccagcttcagctttgttcaacgatgaagcttcagatgaagcttcattgaacgctgctgttgaagaaaccttgaagttcatcgctaaccgtgataaggttgctgttttggttggctcaaagttgcgtgctgctggcgctgaagaagctgctgttaagttcgctgatgctttgggcggcgctgttgctaccatggctgctgctaagtcattcttcccagaagaaaacccacactatatcggcacctcatggggcgaagtttcatatccaggcgttgaaaagaccatgaaggaagctgatgctgttatcgctttggctccagttttcaacgattattcaaccaccggctggaccgatatcccagatccaaagaagttggttttggctgaaccacgttcagttgttgttaacggcgttcgtttcccatcagttcacttgaaggattatttgacccgtttggctcaaaaggtttcaaagaagaccggcgctttggatttcttcaagtcattgaacgctggcgaattgaagaaggctgctccagctgatccatcagctccattggttaacgctgaaatcgctcgtcaagttgaagctttgttgaccccaaacaccaccgttatcgctgaaaccggcgattcatggttcaacgctcaacgtatgaagttgccaaacggcgctcgtgttgaatatgaaatgcaatggggccacatcggctggtcagttccagctgctttcggctatgctgttggcgctccagaacgtcgtaacatcttgatggttggcgatggctcattccaattgaccgctcaagaagttgctcaaatggttcgtttgaagttgccagttatcatcttcttgatcaacaactatggctataccatcgaagttatgatccacgatggcccatataacaacatcaagaactgggattatgctggcttgatggaagttttcaacggcaacggcggctatgattcaggcgctggcaagggcttgaaggctaagaccggcggcgaattggctgaagctatcaaggttgctttggctaacaccgatggcccaaccttgatcgaatgcttcatcggccgtgaagattgcaccgaagaattggttaagtggggcaagcgtgttgctgctgctaactcacgtaagccagttaacaagttgttgtag

SEQ ID NO: 4 - protein, 383 aa, Zymomonas mobilis
MASSTFYIPFVNEMGEGSLEKAIKDLNGSGFKNALIVSDAFMNKSGVVKQVADLLKAQGINSAVYDGVMPNPTVTAVLEGLKILKDNNSDFVISLGGGSPHDCAKAIALVATNGGEVKDYEGIDKSKKPALPLMSINTTAGTASEMTRFCIITDEVRHVKMAIVDRHVTPMVSVNDPLLMVGMPKGLTAATGMDALTHAFEAYSSTAATPITDACALKAASMIAKNLKTACDNGKDMPAREAMAYAQFLAGMAFNNASLGYVHAMAHQLGGYYNLPHGVCNAVLLPHVLAYNASVVAGRLKDVGVAMGLDIANLGDKEGAEATIQAVRDLAASIGIPANLTELGAKKEDVPLLADHALKDACALTNPRQGDQKEVEELFLSAF

SEQ ID NO: 5 - DNA, 1152 bp, Zymomonas mobilis
atggcttcttcaactttttatattcctttcgtcaacgaaatgggcgaaggttcgcttgaaaaagcaatcaaggatcttaacggcagcggctttaaaaatgcgctgatcgtttctgatgctttcatgaacaaatccggtgttgtgaagcaggttgctgacctgttgaaagcacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgccttgaaggctgcgtccatgatcgctaagaatctgaagaccgcttgcgacaacggtaaggatatgccagctcgtgaagctatggcttatgcccaattcctcgctggtatggccttcaacaacgcttcgcttggttatgtccatgctatggctcaccagttgggcggctactacaacctgccgcatggtgtctgcaacgctgttctgcttccgcatgttctggcttataacgcctctgtcgttgctggtcgtctgaaagacgttggtgttgctatgggtctcgatatcgccaatctcggtgataaagaaggcgcagaagccaccattcaggctgttcgcgatctggctgcttccattggtattccagcaaatctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttctaa

SEQ ID NO: 6 - DNA, 1152 bp, Artificial Sequence
  Encoding SEQ ID NO 3, codon-optimized for Lactobacillus paracasei
atggcttcatcaaccttctatatcccattcgttaacgaaatgggcgaaggctcattggaaaaggctatcaaggatttgaacggctcaggcttcaagaacgctttgatcgtttcagatgctttcatgaacaagtcaggcgttgttaagcaagttgctgatttgttgaaggctcaaggcatcaactcagctgtttatgatggcgttatgccaaacccaaccgttaccgctgttttggaaggcttgaagatcttgaaggataacaactcagatttcgttatctcattgggcggcggctcaccacacgattgcgctaaggctatcgctttggttgctaccaacggcggcgaagttaaggattatgaaggcatcgataagtcaaagaagccagctttgccattgatgtcaatcaacaccaccgctggcaccgcttcagaaatgacccgtttctgcatcatcaccgatgaagttcgtcacgttaagatggctatcgttgatcgtcacgttaccccaatggtttcagttaacgatccattgttgatggttggcatgccaaagggcttgaccgctgctaccggcatggatgctttgacccacgctttcgaagcttattcatcaaccgctgctaccccaatcaccgatgcttgcgctttgaaggctgcttcaatgatcgctaagaacttgaagaccgcttgcgataacggcaaggatatgccagctcgtgaagctatggcttatgctcaattcttggctggcatggctttcaacaacgcttcattgggctatgttcacgctatggctcaccaattgggcggctattataacttgccacacggcgtttgcaacgctgttttgttgccacacgttttggcttataacgcttcagttgttgctggccgtttgaaggatgttggcgttgctatgggcttggatatcgctaacttgggcgataaggaaggcgctgaagctaccatccaagctgttcgtgatttggctgcttcaatcggcatcccagctaacttgaccgaattgggcgctaagaaggaagatgttccattgttggctgatcacgctttgaaggatgcttgcgctttgaccaacccacgtcaaggcgatcaaaaggaagttgaagaattgttcttgtcagctttctaa

SEQ ID NO: 7 - protein, 57 aa, Lactococcus lactis
MSTKDFNLDLVSVSKKDSGASPRITSISLCTPGCKTGALMGCNMKTATCNCSIHVSK

SEQ ID NO: 8 - protein, 34 aa, Lactococcus lactis
ITSISLCTPGCKTGALMGCNMKTATCNCSIHVSK

SEQ ID NO: 9 - protein, 57 aa, Lactococcus lactis
MSTKDFNLDLVSVSKKDSGASPRITSISLCTPGCKTGALMGCNMKTATCHCSIHVSK

SEQ ID NO: 10 - protein, 34 aa, Lactococcus lactis
ITSISLCTPGCKTGALMGCNMKTATCHCSIHVSK

SEQ ID NO: 11 - protein, 245 aa, Lactococcus lactis
MRRYLILIVALIGITGLSGCYQTSHKKVRFDEGSYTNFIYDNKSYFVTDKEIPQENVNNSKAKFYKLLIVDMKSEKLLSSSNKNSVTLVLNNIYEASDKSLCMGINDRYYKILPESDKGAVKALRLQNFDVTSDISDDNFVIDKNDSRKIDYMGNIYSISDSTVSDEELGEYQDVLAEVRVFDSVSGKSIPRSEWGRIDKDGSNSKQSRTEWDYGEIHSIRGKSLTEAFAVEINDDFKLATKVGN

SEQ ID NO: 12 - protein, 225 aa, Lactococcus lactis
MQVKIQNLSKTYKEKQVLQDISFDIKSGTVCGLLGVNGAGKSTLMKILFGLISADTGKIFFDGQEKTNNQLGALIEAPAIYMNLSAFDNLKTKALLFGISDKRIHETLEVIGLAETGKKRAGKFSLGMKQRLGIGMAILTEPQFLILDEPTNGLDPDGIAELLNLILKLKAKGVTILISSHQLHEISKVASQIIILNKGKIRYNHANNKEDDIEQLFFKIVHGGM

SEQ ID NO: 13 - protein, 242 aa, Lactococcus lactis
MKRIIASEAIKLKKSGTLRLVLIIPFVTLFIAFLMGGIQIFSVFSIYWWETGFLFLLMSLLFLYDIKSEEQAGNFQNVKWKKLSWKIHLAKMLLIWLRGILASIVLIILLYLVAFVFQGIVVVDFMKVSVALIAILLAASWNLPFIYLIFKWINTYVLLAANTLICLIVAPFVAQTPVWFLLPYTYHYKVTESLLNIKPSGDLLTGKINFSIWEVLLPFGLSIVVTIGVSYLLKGVIEHDKK

SEQ ID NO: 14 - protein, 214 aa, Lactococcus lactis
MIRSECLKLKNSLGFYLVFLFTLLELLTVPIYLAFGRSHVSMTDSSLMIFLFFPLLVTILSILIFEQESLANHFQEINVNKKSSRIWLSKLIVVDFLLFFPSAMIWIITGVSQAVGQQGMMIATASWLMAIFLNHFHLLLTFIINRGGSMIIAIIEILLIIFASNKVLLAAYWCPIALPVNFMITGRCAYLIAAVGWIVLSTIILVALSKKKIR

SEQ ID NO: 15 - protein, 91 aa, Lactobacillus gasseri
MVTKYGRNLGLNKVELFAIWAVLVVALLLTTANIYWIADQFGIHLATGTARKLLDAMASGASLGTAFAAILGVTLPAWALAAAGALGATAA

SEQ ID NO: 16 - protein, 58 aa, Lactobacillus gasseri
IYWIADQFGIHLATGTARKLLDAMASGASLGTAFAAILGVTLPAWALAAAGALGATAA

SEQ ID NO: 17 - protein, 53 aa, Lactobacillus gasseri
MQKLLRIIALISLIAAIISFFIFKINYITYILIGIFIGSGFIYQIRAQGRNRK

SEQ ID NO: 18 - protein, 226 aa, Lactobacillus gasseri
MTRNMFVDIKNVSKNLTDFSLENLNISFEKGEIIGLVGENGAGKTTLLNLISGILKPDKGQILLSSNNIGYCFDALPEPENLTIEQLNTIFTGLLAVWDSDTYFSFVDEFKLPKGKPIGVFSKGMKMQLNVSITLSHNPTLLLLDEVTAGLDPIVRRTVLNTIKEYAISSECIVIMTTHNLEDISDICNRLILLNNGSILLDDNFEDKSSKEIELIFRKTLGYGDM

SEQ ID NO: 19 - protein, 212 aa, Lactobacillus gasseri
MKGLVQKDIYQLTSSWFRPVRIFFVIAVLAAGMIFLKQDSSIILVLLLLLMVNNIQSLFIKDSTNRWLSLLKSLKISTFAVIGSRYITLVVICVCGAILNFVYMLFGMLLFNTSTGIDVLVISSICLWVSLIYGLVIIPFLYAFKQNGLTLAIIIMFSCVALLIKFSSAITKLAYIIHSYSYVQLILIAIVALIGIGIISMVVSYLIVEKEK

SEQ ID NO: 20 - protein, 62 aa, Pediococcus sp.
MKKIEKLTEKEMANIIGGKYYGNGVTCGKHSCSVDWGKATTCIINNGAMAWATGGHQGNHKC

SEQ ID NO: 21 - protein, 44 aa, Pediococcus sp.
KYYGNGVTCGKHSCSVDWGKATTCIINNGAMAWATGGHQGNHKC

SEQ ID NO: 22 - protein, 112 aa, Pediococcus sp.
MNKTKSEHIKQQALDLFTRLQFLLQKHDTIEPYQYVLDILETGISKTKHNQQTPERQARVVYNKIASQALVDKLHFTAEENKVLAAINELAHSQKGWGEFNMLDTTNTWPSQ

SEQ ID NO: 23 - protein, 77 aa, Brochothrix campestris
MKKIEKLTEKEMANIIGGYSSKDCLKDIGKGIGAGTVAGAAGGGLAAGLGAIPGAFVGAHFGVIGGSAACIGGLLGN

SEQ ID NO: 24 - protein, 59 aa, Brochothrix campestris
YSSKDCLKDIGKGIGAGTVAGAAGGGLAAGLGAIPGAFVGAHFGVIGGSAACIGGLLGN

SEQ ID NO: 25 - protein, 61 aa, Brochothrix campestris
MKKIEKLTEKEMANIIGGKINWGNVGGSCVGGAVIGGALGGLGGAGGGCITGAIGSIWDQW

SEQ ID NO: 26 - protein, 43 aa, Brochothrix campestris
KINWGNVGGSCVGGAVIGGALGGLGGAGGGCITGAIGSIWDQW

SEQ ID NO: 27 - protein, 53 aa, Brochothrix campestris
MVKTILFSVVISFVALCNFLIKKDVSSKKKLFLTGSIAVFLIIYDFLWIIFSN

SEQ ID NO: 28 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 29 - DNA, 1548 bp, Saccharomycopsis fibuligera
atgatcagattgacagtctttttgacagcagtttttgctgcagttgctagttgcgtcccggtggaattggacaaaagaaacactggacatttccaagcttattctggatacacagttgccagatcaaatttcactcaatggattcatgagcaaccagctgtttcttggtattatcttttgcaaaacattgattatccagaaggacaatttaaatctgcaaagccaggcgtggtagttgcttctccatccacctcagaacctgactatttttatcaatggaccagagacactgccattacatttctttcgttgattgccgaggttgaagaccatagctttagcaataccacccttgccaaggtcgtggaatactacatcagcaacacctacactttgcaaagagtttcaaacccaagtggaaatttcgacagtcctaaccacgacggtttgggagaaccaaagttcaatgttgacgacaccgcctacacagcttcttggggcagacctcaaaatgatggcccagctttaagagcttatgccatttccagatatttgaatgctgtggccaaacataacaatggcaaattgttgctcgccggccaaaacggaatcccttattctagtgcttctgacatttattggaaaattattaaaccagacttgcaacatgtcagcacccattggagcacctctggctttgatctttgggaagaaaatcaaggaactcatttcttcactgctttggttcaactcaaagctcttagctacggtattcctttgagtaagacttacaacgaccctggctttacttcctggcttgaaaaacaaaaagatgccttgaactcatacatcaactcctctggattcgtcaactcgggtaaaaaacatattgttgaaagcccacaactttcttctagaggcggtttggacagtgccacctacattgctgccttgatcacccatgacattggtgatgatgacacttacactcctttcaacgtggataattcctatgtgctcaattccctatactacttgttggttgacaacaaaaacagatacaagatcaatggcaactacaaagcaggtgctgcggttggaagatatccagaagacgtctacaatggcgttggaactagcgaaggtaacccatggcaattggctactgcctacgctggtcaaactttctacactttggcttacaactctttgaaaaataaaaagaacttggttatagaaaaactcaattacgacctttacaactcctttattgctgacttgtccaagattgactcttcttatgcttccaaagatagtttgactttgacttatggcagcgacaactataaaaatgttatcaaaagtttgctacaatttggtgactctttcttgaaagttctccttgaccatattgatgacaatggccaactcaccgaggaaatcaacagatacactggtttccaagccggcgctgtctccttgacttggagtagtggcagtttgcttagtgcaaacagagctagaaacaaattgattgaacttctttga

SEQ ID NO: 30 - DNA, 1548 bp, Artificial Sequence
  Encoding SEQ ID NO 28, codon-optimized for Saccharomyces cerevisiae
atgatcagattgaccgttttcttgaccgctgtttttgctgctgttgcttcttgtgttccagttgaattggataagagaaacaccggtcatttccaagcttattctggttataccgttgctagatctaacttcacccaatggattcatgaacaaccagctgtttcttggtactacttgttgcaaaacatcgattacccagaaggtcaattcaaatctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtga

(19) United States
(12) Patent Application Publication
(10) Pub. No.: US 2022/0007683 A1
(43) Pub. Date: Jan. 13, 2022

(54) PROCESS FOR MODULATING THE NUTRITIONAL VALUE OF WHOLE STILLAGE AND DISTILLERS PRODUCTS ASSOCIATED THERETO

(71) Applicant: Lallemand Hungary Liquidity Management LLC, Budapest (HU)

(72) Inventors: James L. Steele, Lebanon, NH (US); Brooks Henningsen, Salisbury, NH (US); Jeffery R. Broadbent, Amalga, UT (US); Ekkarat Phrommao, Lebanon, NH (US); Fernanda Cristina Firmino, Atlanta, GA (US)

(21) Appl. No.: 17/372,332

(22) Filed: Jul. 9, 2021

Related U.S. Application Data
(60) Provisional application No. 63/050,588, filed on Jul. 10, 2020.

(51) Int. Cl. A23K 10/38; A23K 10/18; A23K 10/12; A23K 20/147; C12N 1/18

(52) U.S. Cl. CPC A23K 10/38; A23K 10/18; C12N 1/18; A23K 20/147; A23K 10/12

(57) ABSTRACT

Fermentation by-products can be used in feed to provide nutrients to animals. The present disclosure concerns a process for modulating the nutritional content in a whole stillage. The process includes fermenting a biomass in the presence of a recombinant lactic acid bacteria (LAB) cell and a yeast with a biomass and recuperating the whole stillage once the fermentation has been completed. The recombinant LAB is capable of expressing one or more first heterologous enzyme for converting the biomass into the fermentation product.

### CROSS-REFERENCE TO RELATED APPLICATIONS AND DOCUMENTS

This application claims priority from U.S. provisional patent application 63/050,588 filed on Jul. 10, 2020.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 580127_43201_SEQUENCE_LISTING.txt. The text file is 45.3 KB, was created on Jul. 7, 2021, and is being submitted electronically via EFS-Web.

### TECHNOLOGICAL FIELD

The present disclosure concerns the modulation of the nutritional content of distillers products by using a recombinant lactic acid bacteria host cell during fermentation.

### BACKGROUND

Distillers products are obtained after the removal of ethanol by distillation of the yeast fermentation of grain or grain mixtures. These products include dried solubles (DS) or syrup, distillers wet grains, distillers dried grains (DDG), distillers wet grain with solubles, distillers dried grains with solubles (DDGS), and condensed distillers' solubles (CDS) or concentrated syrup which are used as components of feed for animals. Protein and fat are an important nutrient in animal feed, and so are used as an important indicator of distillers' product's quality. For this reason, process changes that increase or chemically alter the protein content serve to enhance distiller's product quality, particularly if such process changes enrich the relative concentrations of essential amino acids in the protein fraction.

It would be highly desirable to provide a process for modulating the nutritional value of a distiller's product to provide a better feed or feed additive.

### BRIEF SUMMARY

The present disclosure concerns the use of a lactic acid bacteria (LAB) for modulating the nutritional content of whole stillage. The lactic acid bacteria is used during a fermentation of a biomass with a yeast.

According to a first aspect, the present disclosure provides a process of modulating the nutritional content of a whole stillage obtained after the fermentation of a biomass. The process comprises (a) contacting a recombinant lactic acid bacteria (LAB) cell, a yeast and the biomass under conditions to cause the conversion of at least in part of the biomass into a fermentation product and to obtain a fermented biomass comprising the whole stillage and the fermentation product; and (b) separating the whole stillage from the fermentation product. In the process of the present disclosure, the recombinant LAB host cell is capable of expressing one or more first heterologous enzyme for converting the biomass into the fermentation product. Furthermore, the whole stillage obtained after step (b) has a different nutritional content than a control whole stillage submitted to step (a) in the absence of the recombinant LAB host cell. In an embodiment, the whole stillage has, when compared to the control whole stillage: an increase in protein content; a different amino acid profile; an increase in fiber content; and/or an increase in lipid content. In another embodiment, the biomass comprises starch. In such embodiment, the whole stillage can have, when compared to the control whole stillage, a decrease in starch content. In some embodiments, the biomass comprises or is obtained from corn. In yet another embodiment, the fermentation product comprises or is ethanol. In such embodiment, the one or more first heterologous enzyme comprises: a polypeptide having pyruvate decarboxylase activity; and/or a polypeptide having alcohol dehydrogenase activity. In an embodiment, the recombinant LAB host cell has a decreased lactate dehydrogenase activity when compared to a corresponding native LAB host cell. In some embodiments, the recombinant LAB host cell has at least one inactivated native gene coding for a lactate dehydrogenase. In yet another embodiment, the at least one native gene coding for the lactate dehydrogenase is Idh1, Idh2, Idh3 or Idh4. In another embodiment, the recombinant LAB host cell has a decreased mannitol dehydrogenase activity compared to a corresponding native LAB host cell. In some embodiments, the recombinant LAB host cell has at least one inactivated native gene coding for a mannitol-1-phosphate 5-dehydrogenase. In specific embodiments, the at least one native gene coding for the mannitol-1-phosphate 5-dehydrogenase is mltD1 or mltD2. In yet another embodiment, the biomass comprises one or more bacteriocin and the recombinant LAB host cell expresses one or more second polypeptide conferring immunity to the one or more bacteriocin. In still another embodiment, the recombinant LAB host cell expresses the one or more bacteriocin. In some embodiments, the biomass comprises one or more antibiotic and the recombinant LAB host cell expresses one or more third heterologous polypeptide conferring resistance to the one or more antibiotic or is adapted to be resistant to the antibiotic. In a further embodiment, the recombinant LAB host cell expresses one or more fourth polypeptide having proteolytic activity (which can be a native polypeptide or a heterologous polypeptide). In some further embodiments, the one or more fourth polypeptide having proteolytic activity comprises a fourth heterologous polypeptide having proteolytic activity. In embodiments in which the recombinant LAB host cell expresses one or more fourth polypeptide having proteolytic activity, the proteolytic activity associated with the recombinant LAB host cell is higher than the proteolytic activity associated with a control LAB cell lacking the ability to express the one or more fourth polypeptide. In another embodiment, the recombinant LAB expresses one or more fifth polypeptide involved in the metabolism one or more amino acid (which can be a native polypeptide or a heterologous polypeptide). In a specific embodiment, the one or more fifth polypeptide is a heterologous polypeptide involved in the metabolism the one or more amino acid. In still another embodiment, the one or more amino acid comprises an essential amino acid, such as, for example, glutamate/gamma-amino butyrate. In such embodiment, the one or more fifth polypeptide comprises one or more polypeptide involved in the metabolism of glutamate/gamma-amino butyrate, such as, for example, a glutamate decarboxylase and/or a glutamate/gamma-amino butyrate (GABA) transporter. In an embodiment, the recombinant LAB host cell is from the genus Lactobacillus sp. and in yet another embodiment, the recombinant LAB host cell is from the species Lactobacillus paracasei. In an embodiment, the yeast is a recombinant yeast host cell. In still another embodiment, the yeast is from the genus Saccharomyces sp., and can be, for example, from the species Saccharomyces cerevisiae. In yet another embodiment, the process further comprises, at step (b), distilling the fermented biomass to remove the fermentation product from the whole stillage. In an embodiment, the process further comprises centrifuging the fermented biomass to separate a thin stillage from a wet cake. In another embodiment, the process further comprises formulating the wet cake in distillers wet grains (DWG). In another embodiment, the process further comprises drying the wet cake to obtain distillers dried grains (DDG). In yet another embodiment, the process further comprises evaporating the thin stillage to obtain a syrup. In some embodiment, the process further comprises adding the syrup to the wet cake to obtain distillers wet grains with solubles (DWGS). In yet another embodiment, the process further comprises drying the DWGS to obtain distillers dried grains with solubles (DDGS). In yet another embodiment, the process further comprising drying the syrup to obtain dried solubles (DS).

According to a second aspect, the present disclosure concerns a whole stillage obtainable or obtained by the process described herein and comprising a component of a recombinant LAB host cell defined herein. The present disclosure also provides a composition comprising a whole stillage obtainable or obtained by the process described herein and a component of a recombinant LAB host cell defined herein.

According to a third aspect, the present disclosure concerns distillers wet grains (DWG) obtainable or obtained by the process described herein and comprising a component of a recombinant LAB host cell defined herein. The present disclosure also concerns a composition comprising distillers wet grains (DWG) obtainable or obtained by the process described herein and a component of a recombinant LAB host cell defined herein.

According to a fourth aspect, the present disclosure concerns distillers dried grains (DDG) obtainable or obtained by the process described herein and comprising a component of a recombinant LAB host cell defined herein. The present disclosure also concerns a composition comprising distillers dried grains (DDG) obtainable or obtained by the process described herein and a component of a recombinant LAB host cell defined herein.

According to a fifth aspect, the present disclosure concerns a syrup obtainable or obtained by the process described herein and comprising a component of a recombinant LAB host cell defined herein. The present disclosure also concerns a composition comprising a syrup obtainable or obtained by the process described herein and a component of a recombinant LAB host cell defined herein.

According to a sixth aspect, the present disclosure concerns distillers wet grains with solubles (DWGS) obtainable or obtained by the process described herein and comprising a component of a recombinant LAB host cell defined herein. The present disclosure also concerns a composition comprising distillers wet grains with solubles (DWGS) obtainable or obtained by the process described herein and a component of a recombinant LAB host cell defined herein.

According to a seventh aspect, the present disclosure concerns distillers dried grains with solubles (DDGS) obtainable or obtained by the process described herein and comprising a component of a recombinant LAB host cell defined herein. The present disclosure also concerns a composition comprising distillers dried grains with solubles (DDGS) obtainable or obtained by the process described herein and a component of a recombinant LAB host cell defined herein.

According to an eighth aspect, the present disclosure concerns dried solubles (DS) obtainable or obtained by the process described herein and comprising a component of a recombinant LAB host cell defined herein. The present disclosure also concerns a composition comprising dried solubles (DS) obtainable or obtained by the process described herein and a component of a recombinant LAB host cell defined herein.

According to a ninth aspect, the present disclosure concerns a feed comprising distillers wet grains as defined herein, distillers dried grains as defined herein, a syrup as defined herein, distillers wet grains with solubles as defined herein, distillers dried grains with solubles as herein, dried solubles as described herein and/or the composition as described herein.

### BRIEF DESCRIPTION OF THE DRAWINGS

Having thus generally described the nature of the invention, reference will now be made to the accompanying drawing, showing by way of illustration, a preferred embodiment thereof, and in which:

FIG. 1 provides a schematic overview of an embodiment of corn ethanol production process for the generation of ethanol and distillers' co-products. CDS=Condensed distillers' solubles, DWGS=distillers wet grains with solubles, DDGS=distillers dried grains with solubles, DWG=distillers wet grains, DDG=distillers dried grains. This is an adaptation of FIG. 1 of Saunders et al. (2013).

### DETAILED DESCRIPTION

The present disclosure concerns the use a recombinant lactic acid bacteria (LAB) host cell to modulate the nutritional content of a whole stillage of a biomass being fermented. In some embodiments, the recombinant LAB host cell can be used to increase the protein content of the whole stillage, provide a different amino acid profile to the whole stillage, increase in fiber content of the whole stillage, increase the lipid content of the whole stillage and/or, when the biomass comprises starch, decrease the starch content of the whole stillage.

Fermentation products, such as ethanol, are obtained from fermenting a biomass, such as a biomass which can be obtained from a grain (including but not limited to corn, barley, rye and wheat). Once the fermentation products are removed from the fermented biomass, it is possible to recuperate some by-products (e.g., distillers products) which can be used as animal feed or a supplement for animal feed. Typically, the starting material (which can be grains and include starch) is first ground in a dry-grind or wet-milling process. The ground starting material can be submitted to a cooking step and/or to an enzymatic starch-degrading step to breakdown the starchy material into fermentable sugars. The fermentable sugars are then converted directly or indirectly into the desired fermentation product using a fermenting organism (e.g., a yeast for example). Liquid fermentation products are recovered from the fermented biomass (often referred to as “beer mash”), e.g., by distillation, which separate the desired fermentation product from other liquids and/or solids. The remaining fraction is referred to as “whole stillage”. The whole stillage can be dewatered and separated into a solid and a liquid phase, e.g., by centrifugation. The solid phase of the whole stillage is referred to as a “wet cake” (or “wet grains”) and the liquid phase (supernatant) is referred to as “thin stillage”. The wet cake can be used without further evaporation as distillers wet grains (DWG). Dewatered wet cake can be dried to provide distillers dried grains (DDG). Thin stillage is typically evaporated to provide a condensate or a syrup or may alternatively be recycled directly to the slurry tank. Condensate may either be forwarded to a methanator before being discharged or may be recycled to the slurry tank. The syrup may be blended into DDG or added to the wet cake before drying to produce distillers wet grains with solubles (DWGS) and optionally dried to provide distillers dried grain with solubles (DDGS). The syrup may be dried to provide dried solubles.

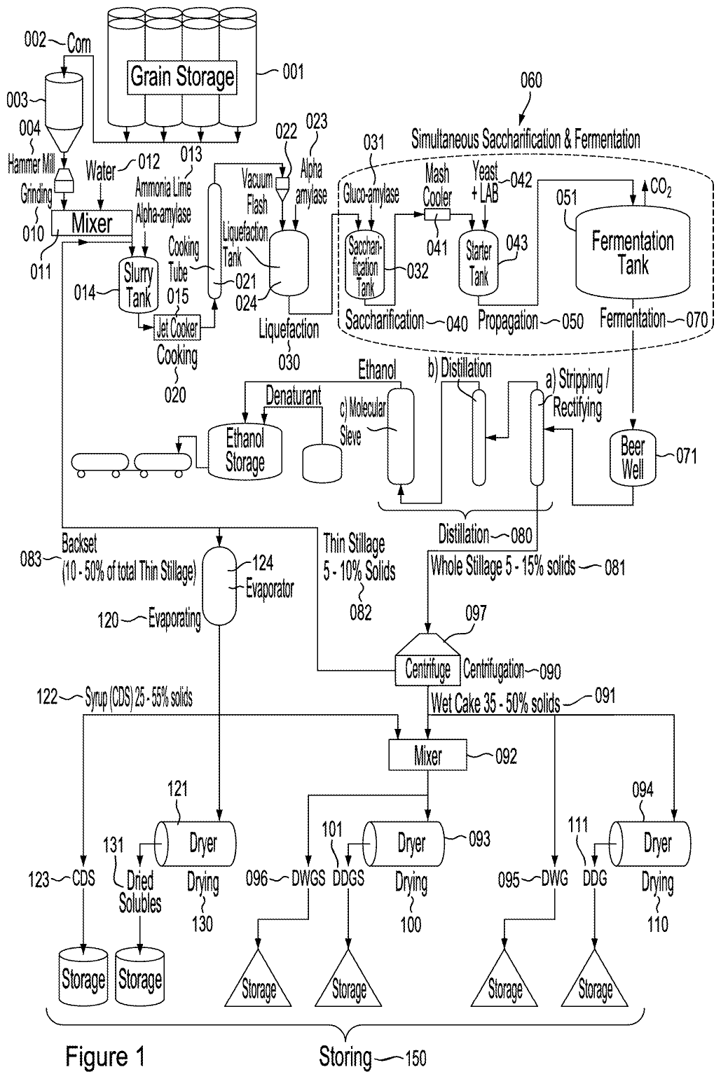

An embodiment of a process for making distillers products from ground corn is shown in FIG. 1. The process can include providing ground corn or steps for making ground corn. Prior to being ground, corn 002 can be optionally stored in a grain storage silo 001 and transferred to a grain storage tank 003. In the embodiment shown in FIG. 1, corn 002 is submitted to a grinding step 010 using a mill 004 (a Hammer mill is shown on FIG. 1 and it is understood that the corn can be substituted by any other suitable mill). The ground corn can be transferred to a mixer 011 and optionally be combined with water 012 to provide a corn mixture which can be used in downstream operations.

The process can also include providing a liquified mixture obtained from corn or can include a step of cooking 020 and liquifying 030 the corn mixture to obtain such liquefied mixture. In the embodiment shown in FIG. 1, the corn mixture is supplemented with a source of alpha-amylase activity 013 (optionally in combination with amonia and lime) in a slurry tank 014. The source of alpha-amylase activity 013 can be a polypeptide having alpha-amylase provided in a purified form and/or may be provided by a recombinant microbial host cell (such as a recombinant yeast host cell) expressing or having expressed the polypeptide having alpha-amylase activity in a recombinant form. The slurry tank can also receive the backset 083 obtained from the thin stillage 082. The content of the slurry tank 014 can be submitted to a cooking step 020 to gelatinize, at least in part, the starch material of the corn mixture. In the embodiment shown in FIG. 1, a jet cooker 015 and a cooking tube 021 are used during cooking step 020 to heat the mixture present in the slurry tank 014. However, it will be recognized that other means of cooking the mixture corn can also be used during cooking step 020. The cooked corn mixture can be introduced into a flash vaccum 022, prior to being transferred to a liquefaction tank 024. In the liquefaction tank 024, at step 030, it is possible to add a source of alpha-amylase activity 023. The source of alpha-amylase activity 023 can be a polypeptide having alpha-amylase activity provided in a purified form and/or may be provided by a recombinant microbial host cell (such as a recombinant yeast host cell) expressing or having expressed the polypeptide having alpha-amylase activity in a recombinant form.

The process can include providing a saccharified corn mixture or can include a step of saccharifying the liquefied corn mixture. In the embodiment shown on FIG. 1, a simultaneous saccharification and fermentation process 060 is shown. In such process, the liquefied corn obtained after step 030 can be supplied to another tank (which can be referred to as a saccharification tank 032). In the saccharification tank 032, a source of glucoamylase activity 031 can be added to the liquefied corn to further saccharify (e.g., breakdown the starch molecule) of the liquefied corn mixture. The source of glucoamylase activity 031 can be a polypeptide having glucoamylase activity in a purified form and/or may be provided by a recombinant microbial host cell (such as a recombinant yeast host cell) expressing or having expressed the polypeptide having glucoamylase activity in a recombinant form. Once the saccharification step 040 has been completed, the saccharified mixture can be cooled down using a mash cooler 041 prior to being added to a starter tank 043. In the context of the present disclosure, the content of the starter tank 043 is admixed with a fermenting yeast and a recombinant lactic acid bacteria mixture 042 and cultured under to conditions so as to favor the propagation, at step 050, of the inoculated microbes. After the microbial cells have propagated, the mixture is transferred to fermentation tank 051 to allow the fermentation, at step 070, of the corn mixture and the production of fermentation products and by-products. It is understood that both the yeast and the recombinant LAB host cell have the ability to convert part of the biomass into one or more fermentation products, but that the fermenting yeast cell exhibits the highest activity in converting part of the biomass into the fermentation products/by-products. It is also understood that the presence of the recombinant LAB host cell during the fermentation causes a modulation in the nutritional value of the resulting whole stillage (e.g., a fermentation by-product).

During the fermentation step 070, fermentation products (CO₂ for example) can be removed (actively or passively) from the fermentation tank 051. Other fermentation products (ethanol for example) can only be obtained from downstream operations of the fermented mixture once fermentation has been completed. In the embodiment shown in FIG. 1, the fermented mixture can be transferred to a beer well 071 and be submitted to a distillation step 080 (which can include a stripping/recycling step 080a, a distillation step 080b and a molecular sieving step 080c) to separate the fermentation product (which can be an alcohol like ethanol) from whole stillage 081.

As shown on FIG. 1, the process can use corn as a fermentable biomass. The biomass that can be fermented includes any type of biomass known in the art and described herein. For example, the biomass can include, but is not limited to, starch (including starch material derived from grains), sugar and lignocellulosic materials. Starch materials can include, but are not limited to, mashes such as corn, wheat, rye, barley, rice, or milo. Starch, when present, can be provided in a raw or a gelatinized form. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and pro line -rich proteins).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASO), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

Further examples of biomasses can comprise, for example, a fruit (apple, grape, pears, plums, cherries, peaches), a plant (sugar cane, agava, cassava, ginger), a starchy material (rice, rye, corn, Sorghum, millet, barley, wheat) or a derived product (grape must, apple mash, malted grain, crushed fruit, fruit puree, fruit juice, fruit must, plant mash, gelatinized and saccharified starch from different plant origins as corn, rye, wheat, barley). In another embodiment, the biomass can be or comprise a starchy material. In the context of the present disclosure, a “starchy material” refers to a material that contains starch that could be converted into alcohol by a yeast during alcoholic fermentation. Starchy material could be for example, gelatinized and saccharified starch from cereals, grains (wheat, barley, rice, buckwheat) or grain derived-products (malted grain or a wort) or vegetable (potatoes, beets). In yet another embodiment, the biomass can be or comprise, but is not limited to, malt, barley, wheat, rye, oats, corn, buckwheat, millet, rice, or sorghum.

The biomass can be supplemented with one or more compound capable of limiting or inhibiting the growth of contaminating bacterial cells. Such embodiment may be advantageous when bacterial contamination is expected or suspected of occurring prior to or during the fermentation. In an embodiment, the one or more compound capable of limiting or inhibiting the growth of a contaminating bacterial cell comprises at least one bacteriocin (alone or in combination with at least one antibiotic). In some embodiments, the at least one bacteriocin comprises one or more bacteriocin from Gram-negative bacteria. The bacteriocin from Gram-negative bacteria which can be used also or in combination with one or more additional bacteriocin. Bacteriocins from Gram-negative bacteria include, but are not limited to, microcins, colicin-like bacteriocins and tailocins. In some embodiments, the at least one bacteriocin comprises one or more bacteriocin from Gram-positive bacteria. The bacteriocin from Gram-positive bacteria which can be used also or in combination with one or more additional bacteriocin. Bacteriocins from Gram-positive bacteria include, but are not limited to, class I bacteriocins (such as, for example nisin A and/or nisin Z), class II bacteriocins, including class IIa (such as, for example, pediocin) and IIb (such as, for example, brochocin for example) bacteriocins, class III bacteriocins, class IV bacteriocins and circular bacteriocins (such as, for example, gassericin). Known bacteriocins include, but are not limited to, acidocin, actagardine, agrocin, alveicin, aureocin, aureocin A53, aureocin A70, bisin, carnocin, carnocyclin, caseicin, cerein, circularin A, colicin, curvaticin, divercin, duramycin, enterocin, enterolysin, epidermin/gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin S, mutacin, nisin A, nisin Z, paenibacillin, planosporicin, pediocin, pentocin, plantaricin, pneumocyclicin, pyocin, reutericin 6, sakaci, salivaricin, sublancin, subtilin, sulfolobicin, tasmancin, thuricin 17, trifolitoxin, variacin, vibriocin, warnericin and warnerin.

In an embodiment, the one or more compound capable of limiting or inhibiting the growth of a bacterial cell comprises at least one antibiotic (alone or in combination with at least one bacteriocin). Antibiotics are commonly classified based on their mechanism of action, chemical structure, or spectrum of activity. Most target bacterial functions or growth processes. Those that target the bacterial cell wall (penicillins and cephalosporins) or the cell membrane (polymyxins), or interfere with essential bacterial enzymes (rifamycins, lipiarmycins, quinolones, and sulfonamides) have bactericidal activities. Protein synthesis inhibitors (macrolides, lincosamides, and tetracyclines) are usually bacteriostatic (with the exception of bactericidal aminoglycosides). Further categorization is based on their target specificity. “Narrow-spectrum” antibiotics target specific types of bacteria, such as gram-negative or gram-positive, whereas broad-spectrum antibiotics affect a wide range of bacteria. Additional antibiotic classes include, but are not limited to: cyclic lipopeptides (such as daptomycin), glycylcyclines (such as tigecycline), oxazolidinones (such as linezolid), and lipiarmycins (such as fidaxomicin). In an embodiment, the antibiotic comprises or is a beta lactam, such as penicillin. In another embodiment, the antibiotic comprises or is streptogramin, such as virginiamycin. In another embodiment, the antibiotic comprises or is an aminoglycoside, such as streptomycin. In yet a further embodiment, the antibiotic comprises or is a macrolide, such as, for example, erythromycin. In still another embodiment, the antibiotic comprises or is a polyether, such as monensin.

The fermented product can be an alcohol, such as, for example, ethanol, isopropanol, n-propanol, 1-butanol, methanol, acetone and/or 1, 2 propanediol. In an embodiment, the biomass or substrate to be hydrolyzed is a lignocellulosic biomass and, in some embodiments, it comprises starch (in a gelatinized or raw form). In the process of the present disclosure, the yeast cells can be first contacted with the biomass. Alternatively, the recombinant LAB host cells can first be contacted with the biomass and the yeast can then be added therein. Also, in some embodiments, both the yeasts and the recombinant LAB host cells can be contacted simultaneously with the biomass. Further, in additional embodiments, the yeasts can first be contacted with the biomass and the recombinant LAB host cells can then be added therein.

The fermentation process can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C. In some embodiments, the process can be conducted at temperatures above about 30° C., about 31° C., about 32° C., about 33° C., about 34° C., about 35° C., about 36° C., about 37° C., about 38° C., about 39° C., about 40° C., about 41° C., about 42° C., or about 50° C.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, or at least about 500 mg per hour per liter.

Once fermentation has been completed and that some of the fermentation product has been removed from the fermented biomass, the solid and the liquid fraction of whole stillage 081 can be separated. This can be achieved, as shown in FIG. 1, by submitting whole stillage 081 to a centrifuging step 090 using a centrifuge 097. It will be recognized that it is possible to separate the solid and the liquid fractions of whole stillage using other means such as, for example, filtration. The solid fraction of whole stillage 081 is referred to as a wet cake 091. The wet cake 091 can be used without further modification as a feed or a feed additive as distillers wet grains (DWG) 095. Alternatively, the wet cake 091 can be submitted to a drying step 110 using a dryer 094 to provide dried distillers grains (DDG) 111. The DWG and the DDG can be used as a feed or a feed additive. The DWG and the DDG can optionally be stored at step 150.

The liquid fraction of whole stillage 081 is referred to as thin sillage 082. Thin sillage 082 can be submitted to an evaporating step 120 to provide a syrup 122, using, for example, an evaporator 124. The syrup 122 can be added to the wet cake 091, in a mixer 092, to make distillers wet grains with solubles (DWGS) 096. Alternatively, the wet cake 091 supplemented with the syrup 122 can be dried, at step 100, using a dryer 093 to provide dried distillers grains with solubles (DDGS) 101. The DWGS and the DDGS can be used as a feed or a feed additive. The DWGS and the DDGS can optionally be stored at step 150.

The syrup 122 can be used without further modifications as condensed distillers solubles (CDS) 123. The syrup 122 can also be dried at step 130 using dryer 121 to obtain dried solubles (DS) 131. The CDS and the DS can be used as a feed or a feed additive. The CDS and the DS can optionally be stored at step 150.

As indicated above, the recombinant LAB host cell is used with a yeast (e.g., a fermenting yeast, which can, in some embodiment be a recombinant yeast host cell) to convert the biomass into a fermentation product/by-product (such as ethanol). These recombinant microbial (bacterial and yeast) cells can be obtained by introducing one or more genetic modifications in a corresponding native (parental) microbial host cell. When the genetic modification is aimed at reducing or inhibiting the expression of a specific targeted gene (which is endogenous to the host cell), the genetic modifications can be made in one or all copies of the targeted gene(s). When the genetic modification is aimed at increasing the expression of a specific targeted gene, the genetic modification can be made in one or multiple genetic locations. In the context of the present disclosure, when recombinant microbial cells are qualified as being “genetically engineered”, it is understood to mean that they have been manipulated to either add at least one or more heterologous or exogenous nucleic acid residue and/or remove at least one endogenous (or native) nucleic acid residue. In some embodiments, the one or more nucleic acid residues that are added can be derived from a heterologous cell or the recombinant cell itself. In the latter scenario, the nucleic acid residue(s) is (are) added at a genomic location which is different than the native genomic location. Alternatively or in combination, one or more additional copy of a native gene at is native genomic location is also considered to be a heterologous nucleic acid molecule. The genetic manipulations did not occur in nature and are the results of in vitro manipulations of the native yeast or bacterial host cell.

When expressed in recombinant microbial cells, the heterologous polypeptides described herein are encoded on one or more heterologous nucleic acid molecule. The term “heterologous” when used in reference to a nucleic acid molecule (such as a promoter, a terminator or a coding sequence) refers to a nucleic acid molecule that is not natively found in the microbial cell. “Heterologous” also includes a native coding region, or portion thereof, that is removed from the source organism and subsequently reintroduced into the source organism in a form that is different from the corresponding native gene. This form can be, for example, the introduction of at least one copy of a native gene at a location which is different from its native location and/or the introduction of at least one additional copy of a native gene at its native location. The heterologous nucleic acid molecule is purposively introduced into the recombinant microbial cell. The term “heterologous” as used herein also refers to an element (nucleic acid or protein) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications). With respect to nucleic acid molecules, the term “heterologous” also refers to corresponding degenerate sequences capable of encoding a polypeptide having the same amino acid sequence.The term “heterologous” when used in reference to a polypeptide (or a protein) refers to a polypeptide encoded by the heterologous nucleic acid molecule. The term “heterologous” is also used synonymously herein with the term “exogenous”.

When a heterologous nucleic acid molecule is present in the recombinant microbial cell, it can be integrated in the recombinant microbial host cell's chromosome. The term “integrated” as used herein refers to genetic elements that are placed, through molecular biology techniques, into the chromosome of a recombinant microbial host cell. For example, genetic elements can be placed into the chromosomes of the microbial cell as opposed to in a vector such as a plasmid carried by the recombinant microbial host cell. Methods for integrating genetic elements into the chromosome of a recombinant microbial host cell are well known in the art and include homologous recombination. The heterologous nucleic acid molecule can be present in one or more copies in the recombinant microbial host cell's chormosomes. Alternatively, the heterologous nucleic acid molecule can be independently replicating from the microbial cell's chromosome. In such embodiment, the nucleic acid molecule can be stable and self-replicating.

In some embodiments, heterologous nucleic acid molecules which can be introduced into the recombinant microbial cells are codon-optimized with respect to the intended recipient recombinant microbial host cell (e.g., bacterial or yeast for example). As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

In some embodiments, heterologous nucleic acid molecules which can be introduced into the recombinant microbial cells are codon-optimized with respect to the intended recipient recombinant microbial cell so as to limit or prevent homologous recombination with the corresponding native gene.

The heterologous nucleic acid molecules of the present disclosure comprise a coding region for the one or more heterologous polypeptides to be expressed by the recombinant microbial cell. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing sites, effector binding sites and stem-loop structures. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise a non-coding region, for example a transcriptional and/or translational control regions. “Transcriptional and translational control regions” are DNA regulatory regions, such as promoters, enhancers, terminators, and the like, that provide for the expression of a coding region in a microbial cell. In eukaryotic cells, polyadenylation signals are control regions.

The heterologous nucleic acid molecule can be introduced in the recombinant microbial host cell using a vector. A “vector,” e.g., a “plasmid”, “cosmid” or “artificial chromosome” (such as, for example, a bacterial or yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a microbial cell.

In the heterologous nucleic acid molecule described herein, the promoter and the nucleic acid molecule coding for the one or more polypeptides can be operatively linked to one another. In the context of the present disclosure, the expressions “operatively linked” or “operatively associated” refers to fact that the promoter is physically associated to the nucleotide acid molecule coding for the one or more polypeptide in a manner that allows, under certain conditions, for expression of the one or more polypeptide from the nucleic acid molecule. In an embodiment, the promoter can be located upstream (5′) of the nucleic acid sequence coding for the one or more polypeptide. In still another embodiment, the promoter can be located downstream (3′) of the nucleic acid sequence coding for the one or more polypeptide. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the one or more polypeptide. The promoters can be located, in view of the nucleic acid molecule coding for the one or more polypeptide, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be heterologous to the nucleic acid molecule encoding the one or more polypeptide. The promoter can be heterologous or derived from a strain being from the same genus or species as the microbial cell. In an embodiment, the promoter is derived from the same genus or species of the microbial cell and the heterologous polypeptide is derived from different genus.

In some embodiments, the present disclosure concerns the expression of one or more heterologous polypeptide, a variant thereof or a fragment thereof in a recombinant microbial host cell. The polypeptide “variants” have at least 50%, 55%, 60%, 65%, 70%, 75%, 80%, 85%, 90%, 95%, 96%, 97%, 98% or 99% identity to the heterologous polypeptide described herein as well as exhibit the biological activity associated with the heterologous polypeptide. In embodiments in which the heterologous polypeptide is a pyruvate decarboxylase, a variant pyruvate decarboxylase must exhibit pyruvate decarboxylase activity. In embodiments in which the heterologous polypeptide is an alcohol dehydrogenase, a variant alcohol dehydrogenase must exhibit alcohol dehydrogenase activity. In an embodiment, the variant polypeptide exhibits at least 50%, 55%, 60%, 65%, 70%, 75%, 80%, 85%, 90%, 95%, 96%, 97%, 98% or 99% of the biological activity of the wild-type heterologous polypeptide. A variant comprises at least one amino acid difference when compared to the amino acid sequence of the native polypeptide. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant heterologous polypeptides described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the polypeptide can be a conservative variant or an allelic variant. As used herein, a conservative variant refers to alterations in the amino acid sequence that do not adversely affect the biological functions of the polypeptide. A substitution, insertion or deletion is said to adversely affect the polypeptide when the altered sequence prevents or disrupts a biological function associated with the polypeptide. For example, the overall charge, structure or hydrophobic-hydrophilic properties of the polypeptide can be altered without adversely affecting its biological activity. Accordingly, the amino acid sequence can be altered, for example to render the polypeptide more hydrophobic or hydrophilic, without adversely affecting the biological activities of the polypeptide.

The heterologous polypeptide can be a fragment of the native heterologous polypeptide or fragment of a variant of the polypeptide which exhibits the biological activity of the heterologous polypeptide or the variant. In an embodiment, the fragment polypeptide exhibits at least 50%, 55%, 60%, 65%, 70%, 75%, 80%, 85%, 90%, 95%, 96%, 97%, 98% or 99% of the biological activity of the heterologous polypeptide or variant thereof. In embodiments in which the heterologous polypeptide is a pyruvate decarboxylase, a fragment of the pyruvate decarboxylase must exhibit pyruvate decarboxylase activity. In embodiments in which the heterologous polypeptide is an alcohol dehydrogenase, a fragment of the alcohol dehydrogenase must exhibit alcohol dehydrogenase activity. Polypeptide “fragments” have at least at least 100, 200, 300, 400, 500 or more consecutive amino acids of the polypeptide or the polypeptide variant. A fragment comprises at least one less amino acid residue when compared to the amino acid sequence of the polypeptide and still possess the biological activity of the full-length enzyme. In some embodiments, the “fragments” have at least 50%, 55%, 60%, 65%, 70%, 75%, 80%, 85%, 90%, 95%, 96%, 97%, 98% or 99% identity to the heterologous polypeptides described herein. In some embodiments, fragments of the polypeptides can be employed for producing the corresponding full-length polypeptide by peptide synthesis. Therefore, the fragments can be employed as intermediates for producing the full-length polypeptides.

In some additional embodiments, the present disclosure also provides expressing a polypeptide encoded by a gene ortholog of a gene known to encode the polypeptide. A “gene ortholog” is understood to be a gene in a different species that evolved from a common ancestral gene by speciation. In the context of the present disclosure, a gene ortholog encodes a polypeptide exhibiting the same biological function than the native polypeptide.

In some further embodiments, the present disclosure also provides expressing a protein encoded by a gene paralog of a gene known to encode the polypeptide. A “gene paralog” is understood to be a gene related by duplication within the genome. In the context of the present disclosure, a gene paralog encodes a polypeptide that could exhibit additional biological function than the native polypeptide.

### Recombinant Lactic Acid Bacteria (LAB) Cell

LAB are a group of Gram-positive bacteria, non-respiring non-spore-forming, cocci or rods, which produce lactic acid as the major end product of the fermentation of carbohydrates. Bacterial genus of LAB include, but are not limited to, Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, and Weissella. Bacterial species of LAB include, but are not limited to, Lactococcus lactis, Lactococcus garviae, Lactococcus raffinolactis, Lactococcus plantarum, Oenococcus oeni, Pediococcus pentosaceus, Pediococcus acidilactici, Carnococcus allantoicus, Carnobacterium gallinarum, Vagococcus fessus, Streptococcus thermophilus, Enterococcus phoeniculicola, Enterococcus plantarum, Enterococcus raffinosus, Enterococcus avium, Enterococcus pallens Enterococcus hermanniensis, Enterococcus faecalis, and Enterococcus faecium. In an embodiment, the LAB is a Lactobacillus sp. and, include, without limitation the following genera Lactobacillus delbrueckii group, Paralactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus. In an embodiment, the LAB is a Lactobacillus and, in some additional embodiment, the Lactobacillus species is L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii (including L. delbrueckii subsp. bulgaricus, L. delbrueckii subsp. delbrueckii, L. delbrueckii subsp. lactis), L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. ammesii, L. hamsteri, L. harbinensis, L. hayakitensis, L. helveticus, L. hfigardfi, L. omohiochii, L. iners, L. ingluviei, L. intestinalis, L. jensenii, L. johnsonii, L. kalixensis, L. efiranofaciens, L. kefiri, L. kimchii, L. kitasatonis, L. kunkeei, L. leichmannii, L. lindneri, L. alefermentans, L. mall, L. manihotivorans, L. mindensis, L. mucosae, L. murinus, L. nagelfi, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. aralimentarius, L. paraplantarum, L. pentosus, L. perolens, L. plantarum, L. pontis, L. protectus, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae or L. zymae. In a specific embodiment, the recombinant LAB host cell is from the genus Lactococcus sp. and can be, in a further embodiment, from the species Lactococcus paracasei (which has recently been reclassified as Lacticaseibacillus paracasei).

The recombinant LAB host cell is capable of expressing one or more first heterologous polypeptide for converting the biomass into the fermentation product. This ability is provided by the presence of at least one first heterologous nucleic acid molecule encoding the one or more heterologous polypeptide for converting, at least in part, a biomass into a fermented product in the recombinant LAB host cell. This first heterologous nucleic acid molecule can be expressed in a constitutive fashion or not by the recombinant LAB host cell. In some embodiments, more than one first heterologous nucleic acid molecules can be provided to encode a plurality of polypeptides for converting, at least in part, a biomass into a fermented product. In such embodiments, each first heterologous nucleic acid molecules can include one or more coding sequences corresponding to one or more heterologous polypeptides. In another embodiment, a single first heterologous nucleic acid molecule can encode the one or more heterologous polypeptides.

In an embodiment, the one or more first heterologous polypeptide comprises a pyruvate decarboxylase and/or an alcohol dehydrogenase. When the first recombinant LAB host cell has an intrinsic ability of expressing a pyruvate decarboxylase, the first heterologous nucleic acid molecule can encode a heterologous alcohol dehydrogenase. In such embodiment, it is possible that the first heterologous nucleic acid molecule (same or different molecule) encodes a heterologous pyruvate decarboxylase (to increase the overall pyruvate decarboxylase activity of the recombinant LAB host cell). When the recombinant LAB host cell has an intrinsic ability of expressing an alcohol dehydrogenase, the first heterologous nucleic acid molecule can encode a pyruvate decarboxylase. In such embodiment, it is possible that the first heterologous nucleic acid molecule further encodes a heterologous alcohol dehydrogenase (to increase the overall alcohol dehydrogenase activity of the first recombinant LAB host cell). If the recombinant LAB host cell does not have an intrinsic ability of expressing a pyruvate decarboxylase and an alcohol dehydrogenase, the first heterologous nucleic acid molecule can encode an alcohol dehydrogenase and a pyruvate decarboxylase (on the same or different nucleic acid molecules). The one or more first heterologous nucleic acid molecules can be integrated in the bacterial chromosome or be independently replicating from the bacterial chromosome. The nucleic acid molecules encoding the pyruvate decarboxylase and the alcohol dehydrogenase can be on the same or distinct first heterologous nucleic acid molecules.

In an embodiment, the one or more polypeptide for converting a biomass includes a heterologous pyruvate decarboxylase. In such embodiment, the recombinant LAB host cell includes on a first heterologous nucleic acid molecule a coding sequence for a heterologous pyruvate decarboxylase. As used herein, the term “pyruvate decarboxylase” refers to an enzyme catalyzing the decarboxylation of pyruvic acid to acetaldehyde and carbon dioxide.

In Zymomonas mobilis, the pyruvate decarboxylase gene is referred to as PDC (Gene ID: 33073732) and could be used in the recombinant LAB host cell of the present disclosure. In some additional embodiments, the pyruvate decarboxylase polypeptide can be from Lactobacillus florum (Accession Number WP_009166425.1), Lactobacillus fructivorans (Accession Number WP_039145143.1), Lactobacillus lindneri (Accession Number WP_065866149.1), Lactococcus lactis (Accession Number WP_104141789.1), Carnobacterium gallinarum (Accession Number WP_034563038.1), Enterococcus plantarum (Accession Number WP_069654378.1), Clostridium acetobutylicum (Accession Number NP_149189.1), Bacillus megaterium (Accession Number WP_075420723.1) or Bacillus thuringiensis (Accession Number WP_052587756.1). In the recombinant LAB host cell of the present disclosure, the heterologous pyruvate decarboxylase can have the amino acid of SEQ ID NO: 1, be a variant of SEQ ID NO: 1 (having pyruvate carboxylase activity) or be a fragment of SEQ ID NO: 1 (having pyruvate carboxylase activity). In some specific embodiments, the recombinant LAB host cell of the present disclosure can express a heterologous nucleic acid molecule comprising the nucleic acid sequence of SEQ ID NO: 2, a variant thereof (encoding a polypeptide having pyruvate carboxylase activity), a fragment thereof (encoding a polypeptide having pyruvate carboxylase activity) or a degenerate nucleic acid sequence encoding the polypeptide of SEQ ID NO: 1 (its variants or its fragments). In some specific embodiments, the recombinant LAB host cell of the present disclosure can express a heterologous nucleic acid molecule comprising the nucleic acid sequence of SEQ ID NO: 3, a variant thereof (encoding a polypeptide having pyruvate carboxylase activity), a fragment thereof (encoding a polypeptide having pyruvate carboxylase activity) or a degenerate nucleic acid sequence encoding the polypeptide of SEQ ID NO: 1 (its variants or its fragments).

In an embodiment, the one or more polypeptide for converting a biomass includes a heterologous alcohol dehydrogenase. In such embodiment, the recombinant LAB host cell includes on a first heterologous nucleic acid molecule a coding sequence for a heterologous alcohol dehydrogenase. The nucleic acid sequence encoding the heterologous alcohol dehydrogenase can physically be located on the same or on a distinct nucleic acid molecule as the nucleic acid sequence encoding the pyruvate decarboxylase. As used herein, the term “alcohol dehydrogenase” refers to an enzyme of the EC 1.1.1.1 class. In some embodiments, the alcohol dehydrogenase is an iron-containing alcohol dehydrogenase. The alcohol dehydrogenase that can be expressed in the first recombinant LAB host cell includes, but is not limited to, ADH4 from Saccharomyces cerevisiae, ADHB from 9Zymomonas mobilis, FUCO from Escherichia coli, ADHE from Escherichia coli, ADH1 from Clostridium acetobutylicum, ADH1 from Entamoeba nuttalli, BDHA from Clostridium acetobutylicum, BDHB from Clostridium acetobutylicum, 4HBD from Clostridium kluyveri, DHAT from Citrobacter freundii or DHAT from Klebsiella pneumoniae. In an embodiment, the alcohol dehydrogenase can be ADHB from Zymomonas mobilis (Gene ID: AHJ71151.1), Lactobacillus reuteri (Accession Number: KRK51011.1), Lactobacillus mucosae (Accession Number WP_048345394.1), Lactobacillus brevis (Accession Number WP_003553163.1) or Streptococcus thermophiles (Accession Number WP_113870363.1). In the recombinant LAB host cell of the present disclosure, the alcohol dehydrogenase can have the amino acid of SEQ ID NO: 4, be a variant of SEQ ID NO: 4 (having alcohol dehydrogenase activity) or a fragment of SEQ ID NO: 4 (having alcohol dehydrogenase activity). In some specific embodiments, the recombinant LAB host cell of the present disclosure can express a heterologous nucleic acid molecule comprising the nucleic acid sequence of SEQ ID NO: 5, be a variant of the nucleic acid sequence of SEQ ID NO: 5 (encoding a polypeptide having alcohol dehydrogenase activity), be a fragment of the nucleic acid sequence of SEQ ID NO: 5 (encoding a polypeptide having alcohol dehydrogenase activity) or a degenerate nucleic acid sequence encoding the polypeptide of SEQ ID NO: 4 (its variants or its fragments). In some specific embodiments, the recombinant LAB host cell of the present disclosure can express a heterologous nucleic acid molecule comprising the nucleic acid sequence of SEQ ID NO: 6, be a variant of the nucleic acid sequence of SEQ ID NO: 6 (encoding a polypeptide having alcohol dehydrogenase activity), be a fragment of the nucleic acid sequence of SEQ ID NO: 6 (encoding a polypeptide having alcohol dehydrogenase activity) or a degenerate nucleic acid sequence encoding the polypeptide of SEQ ID NO: 4 (its variants or its fragments).

In some embodiments, it may be advantageous to reduce the lactate dehydrogenase activity in the recombinant LAB host cell to allow or increase the conversion of the biomass into the fermentation product. In such embodiment, the first recombinant LAB host cell can be genetically modified as to decrease its lactate dehydrogenase activity. As used in the context of the present disclosure, the expression “lactate dehydrogenase” refers to an enzyme of the E.C. 1.1.1.27 class which is capable of catalyzing the conversion of pyruvic acid into lactate. The recombinant LAB host cell can thus have one or more gene coding for a protein having lactate dehydrogenase activity which is inactivated (via partial or total deletion of the gene). In bacteria, the Idh1, Idh2, Idh3 and Idh4 genes encode proteins having lactate dehydrogenase activity. Some bacteria may contain as many as six or more such genes (i.e., Idh5, Idh6, etc.). In an embodiment, at least one of the Idh1, Idh2, Idh3 and Idh4 genes, their corresponding orthologs and paralogs is inactivated in the recombinant LAB host cell. In an embodiment, only one of the Idh genes is inactivated in the recombinant LAB host cell. For example, in the recombinant LAB host cell of the present disclosure, only the Idh1 gene can be inactivated. In another embodiment, at least two of the Idh genes are inactivated in the recombinant LAB host cell. In another embodiment, only two of the Idh genes are inactivated in the recombinant LAB host cell. In a further embodiment, at least three of the Idh genes are inactivated in the recombinant LAB host cell. In a further embodiment, only three of the Idh genes are inactivated in the recombinant LAB host cell. In a further embodiment, at least four of the Idh genes are inactivated in the recombinant LAB host cell. In a further embodiment, only four of the Idh genes are inactivated in the recombinant LAB host cell. In a further embodiment, at least five of the Idh genes are inactivated in the recombinant LAB host cell. In a further embodiment, only five of the Idh genes are inactivated in the recombinant LAB host cell. In a further embodiment, at least six of the Idh genes are inactivated in the recombinant LAB host cell. In a further embodiment, only six of the Idh genes are inactivated in the recombinant LAB host cell. In still another embodiment, all of the Idh genes are inactivated in the recombinant LAB host cell.

In some embodiments, it may be advantageous to reduce the mannitol-1-phosphate 5-dehydrogenase activity in the recombinant LAB host cell to allow or increase the conversion of the biomass into the fermentation product In such embodiment, the recombinant LAB host cell can be genetically engineered to decrease its mannitol-1-phosphate 5-dehydrogenase activity. As used in the context of the present disclosure, the expression “mannitol-1-P 5-dehydrogenase” refer to an enzyme of the E.C. 1.1.1.17 class which is capable of catalyzing the conversion of mannitol into fructose-6-phosphate. The recombinant LAB host cell can thus have one or more gene coding for a protein having mannitol dehydrogenase activity which is inactivated (via partial or total deletion of the gene). In bacteria, the mltd1 and mltd2 genes encode proteins having mannitol-1-P 5-dehydrogenase activity. In an embodiment, at least one of the mltd1 and mtld2 genes, their corresponding orthologs and paralogs is inactivated in the recombinant LAB host cell. In an embodiment, only one of the mltd1 and mtld2 genes is inactivated in the recombinant LAB host cell. In another embodiment, both of the mltd1 and mtld2 genes are inactivated in the recombinant LAB host cell.

In some embodiments, the recombinant LAB host cell can express a bacteriocin. In some embodiments, the recombinant LAB host cell can have the intrinsic ability (e.g., an ability that is not conferred by the introduction of a heterologous nucleic acid molecule) to express and produce at least one bacteriocin (e.g., a native bacteriocin). In some embodiments, the recombinant LAB host cell can be genetically modified to express and produce one or more bacteriocin (in addition to the one it already expresses, if any). In such embodiment, the recombinant LAB host cell will include one or more second heterologous nucleic acid molecule encoding the bacteriocin and the polypeptide(s) associated with the immunity to the bacteriocin. The coding sequence for the bacteriocin and for the polypeptide(s) associated with the immunity to the further bacteriocin can be provided on the same or distinct second nucleic acid molecules. The second nucleic acid molecule(s) (which can be heterologous) can be integrated in the bacterial chromosome or be independently replicating from the bacterial chromosome.

In other embodiments, the recombinant LAB host cell can also lack the intrinsic ability to express one or more bacteriocin and can be genetically modified to express and produce one or more bacteriocin (e.g., a recombinant bacteriocin). In such embodiment, the recombinant LAB host cell will include one or more second heterologous nucleic acid molecule encoding the recombinant bacteriocin and its associated immunity polypeptide(s). The coding sequence for the recombinant bacteriocin and for the polypeptide(s) associated with the immunity to the recombinant bacteriocin can be provided on the same or distinct second nucleic acid molecules. In some embodiments, the recombinant LAB host cell can be genetically modified to express and produce more than one recombinant bacteriocin and associated immunity polypeptide(s). In such embodiment, the recombinant LAB host cell will include one or more second heterologous nucleic acid molecule encoding the additional recombinant bacteriocin and/or the polypeptide(s) associated with the immunity to the additional recombinant bacteriocin. The coding sequence for the recombinant bacteriocin and for the polypeptide(s) associated with the immunity to the recombinant bacteriocin can be provided on the same or distinct second nucleic acid molecules. The second nucleic acid molecule(s) (which can be heterologous) can be integrated in the bacterial chromosome or be independently replicating from the bacterial chromosome.

In some embodiments, the recombinant LAB will be cultured in the presence of a bacteriocin it does not express (natively or in a recombinant fashion). For example, the biomass can be supplemented with a purified and exogenous source of a bacteriocin. In such embodiment, the recombinant LAB host cell can be genetically modified to express and produce a polypeptide conferring immunity to the bacteriocin present in the biomass. In such embodiment, the recombinant LAB host cell will include one or more second heterologous nucleic acid molecule encoding a bacteriocin immunity polypeptide(s). When more than one type of bacteriocins are present in the biomass, the coding sequence for the polypeptide(s) associated with the immunity of each bacteriocin can be provided on the same or distinct second nucleic acid molecules. In such embodiments, the recombinant LAB host cell can be genetically modified to express and produce more than one associated bacteriocin immunity polypeptide. In such embodiment, the recombinant LAB host cell will include one or more second heterologous nucleic acid molecule encoding the additional polypeptide(s) associated with the immunity to each the bacteriocin present in the biomass. The coding sequence for the polypeptide(s) associated with the immunity to the bacteriocin(s) can be provided on the same or distinct second nucleic acid molecules. The second heterologous nucleic acid molecule(s) can be integrated in the bacterial chromosome or be independently replicating from the bacterial chromosome.

Bacteriocins are known as a class of peptides and polypeptides exhibiting, as their biological activity, anti-bacterial properties. Bacteriocins can exhibit bacteriostatic or cytotoxic activity. Bacteriocin can be provided as a monomeric polypeptide, a dimer polypeptide (homo- and heterodimers) as well as a circular polypeptide. Since bacteriocin are usually expressed to be exported outside of the cell, they are usually synthesized as pro-polypeptides including a leader sequence, the latter being cleaved upon secretion. The bacteriocin of the present disclosure can be expressed using their native leader sequence or a heterologous leader sequence. It is known in the art that some bacteriocins are modified after being translated to include uncommon amino acids (such as lanthionine, methyllanthionine, didehydroalanine, and/or didehydroaminobutyric acid). The amino acid sequences provided herein for the different bacteriocins do not include such post-translational modifications, but it is understood that a recombinant LAB host cell expressing a bacteriocin from a second heterologous nucleic acid molecule can produce a polypeptide which does not exactly match the amino acid sequence of the different SEQ ID NOs, but the exported bacteriocin can be derived from such amino acid sequences (by post-translational modification).

In some embodiments, the at least one bacteriocin comprises one or more bacteriocin from Gram-negative bacteria. The bacteriocin from Gram-negative bacteria which can be used also or in combination with one or more additional bacteriocin. Bacteriocins from Gram-negative bacteria include, but are not limited to, microcins, colicin-like bacteriocins and tailocins. In some embodiments, the at least one bacteriocin comprises one or more bacteriocin from Gram-positive bacteria. The bacteriocin from Gram-positive bacteria which can be used also or in combination with one or more additional bacteriocin. Bacteriocins from Gram-positive bacteria include, but are not limited to, class I bacteriocins (such as, for example nisin A and/or nisin Z), class II bacteriocins, including class Ila (such as, for example, pediocin) and IIb (such as, for example, brochocin for example) bacteriocins, class III bacteriocins, class IV bacteriocins and circular bacteriocins (such as, for example, gassericin). Known bacteriocins include, but are not limited to, acidocin, actagardine, agrocin, alveicin, aureocin, aureocin A53, aureocin A70, bisin, carnocin, carnocyclin, caseicin, cerein, circularin A, colicin, curvaticin, divercin, duramycin, enterocin, enterolysin, epidermin/gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin S, mutacin, nisin A, nisin Z, paenibacillin, planosporicin, pediocin, pentocin, plantaricin, pneumocyclicin, pyocin, reutericin 6, sakaci, salivaricin, sublancin, subtilin, sulfolobicin, tasmancin, thuricin 17, trifolitoxin, variacin, vibriocin, warnericin and warnerin.

In a specific embodiment, the bacteriocin expressed by the recombinant LAB host cell or encoded by the second heterologous nucleic acid molecule can be a Gram-positive class I bacteriocin. The Gram-positive class I bacteriocin can be the only bacteriocin expressed in the ecombinant LAB host cell or it can be expressed with one or more further bacteriocin. For example, nisin can be the only bacteriocin present in the biomass or produced by the recombinant LAB host cell. In another example, nisin can be in combination with pediocin and brochocin in the biomass or expressed by the recombinant host LAB host cell. In some embodiments, the Gram-positive class I bacteriocin can be nisin A, nisin Z, nisin J (as described in O'Sullivan et al., 2020), nisin H (as described in O'Connor et al., 2015), nisin Q (as described in Fukao et al., 2008) and/or nisin U (as described in Wirawan et al., 2006). Nisin is a bacteriocin natively produced by some strains of Lactococcus lactis. Nisin is a relatively broad-spectrum bacteriocin effective against many Gram-positive organisms as well as spores. In an embodiment, nisin A has the amino acid sequence of SEQ ID NO: 9 (including its native leader sequence), is a variant of the amino acid sequence of SEQ ID NO: 9 (retaining, at least in part, the biological activity of nisin A) or is a fragment of the amino acid sequence of SEQ ID NO: 9 (retaining, at least in part, the biological activity of nisin A). In an embodiment, nisin A has the amino acid sequence of SEQ ID NO: 10 (excluding its native leader sequence), is a variant of the amino acid sequence of SEQ ID NO: 10 (retaining, at least in part, the biological activity of nisin A) or is a fragment of the amino acid sequence of SEQ ID NO: 10 (retaining, at least in part, the biological activity of nisin A). In an embodiment, nisin Z has the amino acid sequence of SEQ ID NO: 7 (including its native leader sequence), is a variant of the amino acid sequence of SEQ ID NO: 7 (retaining, at least in part, the biological activity of nisin Z) or is a fragment of the amino acid sequence of SEQ ID NO: 7 (retaining, at least in part, the biological activity of nisin Z). In an embodiment, nisin Z has the amino acid sequence of SEQ ID NO: 8 (excluding its native leader sequence), is a variant of the amino acid sequence of SEQ ID NO: 8 (retaining, at least in part, the biological activity of nisin Z) or is a fragment of the amino acid sequence of SEQ ID NO: 8 (retaining, at least in part, the biological activity of nisin Z).

In embodiments in which the recombinant LAB host cell produces nisin as the bacteriocin or in which nisin is present in the biomass, the recombinant LAB host cell can possess the machinery for making nisin or can be genetically engineered to express the machinery for making nisin. Polypeptides involved in the production and/or the regulation of production of nisin include, but are not limited to NisA, NisZ, NisJ, NisH, NisQ, NisB, NisT, NisC, NisP, NisR and/or NisK. The one or more polypeptides involved in the production and/or the regulation of production of nisin can be located on the same or a distinct nucleic acid molecule as the one encoding nisin.

In embodiments in which the recombinant LAB host cell produces nisin as the bacteriocin or in which nisin is present in the biomass, the recombinant LAB host cell possesses immunity against nisin or can be genetically engineered to gain immunity against nisin. A polypeptide known to confer immunity or resistance against nisin is Nisl. In an embodiment, Nisl has the amino acid sequence of SEQ ID NO: 11 (as well as functional variants and fragments thereof retaining at least on part their ability to confer immunity against nisin). As such, the second heterologous nucleic acid molecule can further encode Nisl. Additional polypeptides involved in conferring immunity against nisin include, without limitation, NisE (which is a nisin transporter), NisF (which is a nisin transporter) and NisG (which is a nisin permease). As such, the second heterologous nucleic acid molecule can further encode NisE, NisF and/or NisG. In an embodiment, NisE has the amino acid sequence of SEQ ID NO: 13 (as well as functional variants and fragments thereof retaining, at least in part, their ability to transport nisin). In an embodiment, NisF has the amino acid sequence of SEQ ID NO: 12 (as well as functional variants and fragments thereof retaining, at least in part, their ability to transport nisin). In an embodiment, NisG has the amino acid sequence of SEQ ID NO: 14 (as well as functional variants and fragments thereof retaining, at least in part, their ability to transport nisin). The one or more polypeptides involved in the conferring immunity against nisin can be located on the same or on a distinct nucleic acid molecule as the one encoding nisin and/or the polypeptides involved in the production and/or the regulation of production of nisin.

In a specific embodiment, the bacteriocin present in the biomass or expressed by the recombinant LAB host cell can be a Gram-positive class II bacteriocin. The Gram-positive class II bacteriocin can be the only bacteriocin expressed in the ecombinant LAB host cell or it can be expressed with one or more further bacteriocin. Gram-positive class II bacteriocins include two subgroups: class IIA and class IIB bacteriocins. In a specific example, the Gram-positive class IIA bacteriocin can be, without limitation, pediocin (also referred to as the PedA polypeptide). In an embodiment, pediocin has the amino acid sequence of SEQ ID NO: 20 (including its native leader sequence), is a variant of the amino acid sequence of SEQ ID NO: 20 (retaining, at least in part, the biological activity of pediocin) or is a fragment of the amino acid sequence of SEQ ID NO: 20 (retaining, at least in part, the biological activity of pediocin). In an embodiment, pediocin has the amino acid sequence of SEQ ID NO: 21 (excluding its native leader sequence), is a variant of the amino acid sequence of SEQ ID NO: 21 (retaining, at least in part, the biological activity of pediocin) or is a fragment of the amino acid sequence of SEQ ID NO: 21 (retaining, at least in part, the biological activity of pediocin).

In embodiments in which the recombinant LAB host cell produces pediocin as the bacteriocin or in which pediocin is present in the biomass, the recombinant LAB host cell can possess the machinery for making and regulating pediocin production or can be genetically engineered to express the machinery for making and regulating pediocin production. A polypeptide known to confer immunity or resistance against pediocin is PedB. In an embodiment, PedB has the amino acid sequence of SEQ ID NO: 22 (as well as functional variants and fragments thereof retaining at least on part their ability to confer immunity against pediocin). As such, the first recombinant LAB host cell can express PedB or be genetically engineered to express PedB. In some embodiments, the second heterologous nucleic acid molecule can further encode PedB (which can be present on the same nucleic acid molecule encoding PedA or a distinct one).

In a specific example, the Gram-positive class IIB bacteriocin can be, without limitation, brochocin. Brochocin is an heterodimer comprising a BrcA polypeptide and a BrcB polypeptide. In an embodiment, BrcA has the amino acid sequence of SEQ ID NO: 23 (including the pediocin leader sequence), is a variant of the amino acid sequence of SEQ ID NO: 23 (retaining, at least in part, the biological activity of brochocin when forming an heterodimer with BrcB) or is a fragment of the amino acid sequence of SEQ ID NO: 23 (retaining, at least in part, the biological activity of brochocin when forming an heterodimer with BrcB). In an embodiment, BrcA has the amino acid sequence of SEQ ID NO: 24 (excluding its native leader sequence), is a variant of the amino acid sequence of SEQ ID NO: 24 (retaining, at least in part, the biological activity of brochocin when forming an heterodimer with BrcB) or is a fragment of the amino acid sequence of SEQ ID NO: 24 (retaining, at least in part, the biological activity of brochocin when forming an heterodimer with BrcB). In an embodiment, BrcB has the amino acid sequence of SEQ ID NO: 25 (including the pediocin leader sequence), is a variant of the amino acid sequence of SEQ ID NO: 25 (retaining, at least in part, the biological activity of brochocin when forming an heterodimer with BrcA) or is a fragment of the amino acid sequence of SEQ ID NO: 25 (retaining, at least in part, the biological activity of brochocin when forming an heterodimer with BrcA). In an embodiment, BrcB has the amino acid sequence of SEQ ID NO: 26 (excluding its native leader sequence), is a variant of the amino acid sequence of SEQ ID NO: 26 (retaining, at least in part, the biological activity of brochocin when forming an heterodimer with BrcA) or is a fragment of the amino acid sequence of SEQ ID NO: 26 (retaining, at least in part, the biological activity of brochocin when forming an heterodimer with BrcA).

In embodiments in which the recombinant LAB host cell produces brochocin as the bacteriocin or in which brochocin is present in the biomass, the recombinant LAB host cell possesses immunity against brochocin. A polypeptide known to confer immunity or resistance against borchocin is Brcl. In an embodiment, Brcl has the amino acid sequence of SEQ ID NO: 27 (as well as functional variants and fragments thereof retaining at least on part their ability to confer immunity against brochocin). As such, the recombinant LAB host cell can express Brcl or be genetically engineered to express Brcl. In some embodiments, the second heterologous nucleic acid molecule can further encode Brcl (which can be present on the same nucleic acid molecule encoding BrcA/BrcB or a distinct one).

In embodiments in which the bacteriocin expressed by the recombinant LAB host cell is a Gram-positive class II bacteriocin, the recombinant LAB host cell can express a native non-sec dependent secretory machinery and/or include one or more heterologous nucleic acid molecules encoding a native non-sec dependent secretory machinery for exporting the Gram-positive class II bacteriocin. An exemplary component of a non-sec dependent secretory machinery for exporting the Gram-positive class II bacteriocin is PedC (which can also be referred to as BrcD) which can have, in some additional embodiments, GenBank Accession Number WP_005918571, be a variant of GenBank Accession Number WP_005918571 having disulfide isomerase activity or be a fragment of GenBank Accession Number WP_005918571 having disulfide isomerase activity. A further exemplary component of a non-sec dependent secretory machinery for exporting the Gram-positive class II bacteriocin is PedD (which can also be referred to as PapD) which can have, in some additional embodiments, Uniprot Accession Number P36497.1, be a variant of Uniprot Accession Number P36497.1 having ATP-binding and transporting activity or be a fragment of Uniprot Accession Number P36497.1 having ATP-binding and transporting activity.

In some embodiments, the Gram-positive class II bacteriocin, its variants and its fragments can be associated with a sec-dependent leader peptide so as to facilitate its transport outside the recombinant LAB host cell.

In a specific example, the Gram-positive cyclic bacteriocin can be gasserin. In an embodiment, gasserin has the amino acid sequence of SEQ ID NO: 15 (including its native leader sequence), is a variant of the amino acid sequence of SEQ ID NO: 15 (retaining, at least in part, the biological activity of gasserin) or is a fragment of the amino acid sequence of SEQ ID NO: 15 (retaining, at least in part, the biological activity of gasserin). In an embodiment, gasserin has the amino acid sequence of SEQ ID NO: 16 (excluding its native leader sequence), is a variant of the amino acid sequence of SEQ ID NO: 16 (retaining, at least in part, the biological activity of gasserin) or is a fragment of the amino acid sequence of SEQ ID NO: 16 (retaining, at least in part, the biological activity of gasserin). In such embodiment, the recombinant LAB host cell is capable of expressing gasserin which can be expressed from the second heterologous nucleic acid molecule.

In embodiments in which the first recombinant LAB host cell produces gasserin as the bacteriocin or in which gasserin is present in the culture medium, the recombinant LAB host cell can possess the machinery for making or for regulating the production of gasserin or can be genetically engineered to express the machinery for making or for regulating the production of gasserin. Polypeptides involved in the machinery for making gasserin include, without limitations, GaaT (which is a gasserin transporter) and GaaE (which is a gasserin permease). As such, the second heterologous nucleic acid molecule can further encode GaaT and/or GaaE (which can be on the same or on a different nucleic acid molecule than the one encoding gasserin). In an embodiment, GaaT has the amino acid sequence of SEQ ID NO: 18 (as well as functional variants and fragments thereof retaining, at least in part, their ability to transport gasserin). In an embodiment, GaaE has the amino acid sequence of SEQ ID NO: 19 (as well as functional variants and fragments thereof retaining, at least in part, their ability to transport gasserin).

In embodiments in which the recombinant LAB host cell produces gasserin as the bacteriocin or in which gasserin is present in the biomass, the recombinant LAB host cell possesses immunity against gasserin or can be genetically engineered to gain immunity against gasserin. A polypeptide known to confer immunity or resistance against gasserin is Gaal. In an embodiment, Gaal has the amino acid sequence of SEQ ID NO: 17 (as well as functional variants and fragments thereof retaining at least on part their ability to confer immunity against gasserin). As such, the second heterologous nucleic acid molecule can further encode Gaal (which can be on the same or on a different nucleic acid molecule than the one encoding gasserin, GaaT or GaaE).

In embodiments in which the biomass comprises one or more antibiotic, it is important that the viability or the growth of the recombinant LAB host cell is not reduced or slowed due to the presence of such antibiotic. As such, in some embodiments, the recombinant LAB host cell can include one or more further nucleic acid molecule encoding one or more polypeptide involved in conferring resistance to the antibiotic(s) present in the biomass. Alternatively or in combination, the recombinant LAB host cell can be made more resistant towards the antibiotic(s) present in the biomass by being submitted (prior to the fermentation) to an adaptation process. During an adaptation process, the recombinant LAB host cell is submitted to increasing concentrations of the antibiotic for which resistance is sought. In an embodiment, the recombinant LAB host cell comprises one or more genes conferring resistance to a beta lactam, such as penicillin. In another embodiment, the recombinant LAB host cell comprises one or more genes conferring resistance to streptogramin, such as virginiamycin. In another embodiment, the recombinant LAB host cell comprises one or more genes conferring resistance to aminoglycoside, such as streptomycin. In yet a further embodiment, the recombinant LAB host cell comprises one or more genes conferring resistance to a macrolide, such as, for example, erythromycin. In still another embodiment, the recombinant LAB host cell comprises one or more genes conferring resistance to a polyether, such as monensin. In an embodiment, the recombinant LAB host cell is adapted to become more resistant to a beta lactam, such as penicillin. In another embodiment, the recombinant LAB host cell is adapted to become more resistant to streptogramin, such as virginiamycin. In another embodiment, the recombinant LAB host cell com is adapted to become more resistant to aminoglycoside, such as streptomycin. In yet a further embodiment, the recombinant LAB host cell is adapted to become more resistant to a macrolide, such as, for example, erythromycin. In still another embodiment, the recombinant LAB host cell is adapted to become more resistant to a polyether, such as monensin.

In some embodiments, the recombinant LAB host cell can also be capable of expressing a protease (also referred to as a polypeptide having proteolytic activity). In such embodiment, the recombinant LAB host cell can express one or more fourth polypeptide having proteolytic activity. The one or more fourth polypeptide having proteolytic activity can be natively expressed by the recombinant LAB host cell or can be genetically engineered to express a heterologous polypeptide having proteolytic activity. In the latter embodiment, the recombinant LAB host cell can comprise a heterologous nucleic acid molecule encoding the one or more fourth heterologous polypeptide having proteolytic activity. In the embodiment in which the recombinant LAB host cell expresses one or more heterologous fourth polypeptide, the proteolytic activity associated with this recombinant LAB host cell is higher than a control host cell lacking the ability to express the one or more heterologous fourth polypeptide (and lacking the heterologous nucleic acid molecule encoding the one or more fourth polypeptide).

In the context of the present disclosure, the term “protease” (also referred to as “peptidase”) refers to a polypeptide having proteolytic activity (e.g., a proteolytic enzyme). Proteases can be classified into two groups based on the type of proteolytic activity they exhibit: endopeptidases (which include proteinases) and exopeptidases. Endopeptidases exhibit endo-acting peptide bond hydrolase activity, whereas exopeptidases exhibit exo-acting peptide bond hydrolase activity. The proteases that may be secreted by the recombinant LAB host cell can remain associated with the surface of the bacterial cell (and in some embodiments may be anchored or tethered at the surface of the bacterial cell) or they can be independent from the recombinant LAB host cell (e.g., free). The proteases that may be expressed by the recombinant LAB host cell may not be secreted and may be present in the cytoplasm (intracellular).

Proteases can also be classified under E.C. 3.4 and can be derived from a bacterial cell, a plant cell, a yeast cell or a fungal cell. Proteases can be classified according to their catalytic residue: serine proteases (using a serine alcohol), cysteine proteases (using a cysteine thiol), threonine proteases (using a threonine secondary alcohol), aspartic proteases (using an aspartate carboxylic acid), glutamic proteases (using a glutamate carboxylic acid), metalloproteases (using a metal) and asparagine peptide lyases (using an asparagine to perform an elimination reaction). Alternatively, proteases may be classified by the optimal pH in which they are active: acid proteases, neutral proteases and basic proteases. In an embodiment, the protease expressed by the recombinant LAB host cell is a neutral protease. In a further embodiment, the protease expressed by the recombinant LAB host cell is from a Bacillus sp., for example from Bacillus subtilis. In still a further embodiment, the protease expressed by the recombinant LAB can be a NRPE protease (which have, for example, the GenBank accession number AAC24942). In an embodiment, the protease expressed by the recombinant LAB host cell can be an acidic protease.

In some embodiments, the recombinant LAB host cell can have the ability to metabolize (e.g., catabolize or synthesize) one or more amino acid. This ability can be native or can be genetically engineered in the recombinant LAB host cell. In the latter, the recombinant LAB host cell can include a further heterologous nucleic molecule encoding a polypeptide capable of metabolizing the one or more amino acid. In some embodiments, the amino acid is an essential amino acid for an animal, such as, for example, phenylalanine, valine, threonine, tryptophan, methionine, leucine, isoleucine, lysine, and histidine. In a specific embodiment, the essential amino acid is lysine. In some embodiments, the recombinant LAB host cell can have the ability to convert asparagine and/or citric acid into lysine.

In some embodiments, the recombinant LAB host cell has the ability to metabolize glutamate/gamma-amino butyrate. This ability can be native or can be genetically engineered in the recombinant LAB host cell. In the latter, the recombinant LAB host cell can include a fifth heterologous nucleic molecule encoding a polypeptide involved in the metabolism of glutamate/gamma-amino butyrate (e.g., capable of metabolizing or transporting glutamate/gamma-amino butyrate). For example, the recombinant LAB host cell can express a polypeptide capable of metabolizing glutamate/gamma-amino butyrate such as, for example, a glutamate decarboxylase. In another example, the recombinant LAB host cell can expressing a polypeptide capable of transporting glutamate/gamma-amino butyrate, such as, for example, a glutamate/gamma-amino butyrate (GABA) transporter. In some embodiments, the glutamate decarboxylase can be GADA and/or GADB. In additional embodiments, the GABA transporter can be GADC. The gene encoding the polypeptide capable of metabolizing glutamate/gamma-amino butyrate can be obtained from a Lactobacillus sp., such as, for example, a Lactobacillus brevis, Lactobacills reuteri, and/or a Lactobacillys plantarum.

The recombinant LAB host cell can be provided as a cell concentrate. The cell concentrate comprising the recombinant LAB host cell can be obtained, for example, by propagating the recombinant LAB host cell in a culture medium and removing at least one components of the medium comprising the propagated recombinant LAB host cells. This can be done, for example, by dehydrating, filtering (including ultra-filtrating) and/or centrifuging the medium comprising the propagated recombinant LAB host cells. In an embodiment, the recombinant LAB host cell can be provided as a frozen concentrate in the combination.

### Yeast Cell

The recombinant LAB host cell of the present disclosure is used in combination with a yeast cell to convert the biomass into the fermentation product/by-product. In the context of the present disclosure, the yeast cell is considered to be a fermenting yeast cell because it is responsible for the majority of the conversion of the biomass into the fermentation product/by-product. The yeast cell can be a wild-type native yeast cell or a can be recombinant yeast host cell. In some embodiments, the yeast cell can be a population comprising both a wild-type native yeast cell and a recombinant yeast host cell.

Suitable yeast cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast cell is Saccharomyces cerevisiae. In some embodiments, the yeast cell can be an oleaginous yeast cell. For example, the oleaginous yeast cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the yeast cell can be an oleaginous microalgae host cell (e.g., for example, from the genus Thraustochytrium or Schizochytrium). In an embodiment, the yeast cell is from the genus Saccharomyces and, in some embodiments, from the species Saccharomyces cerevisiae.

In a specific embodiment, the yeast cell can have one or more genetic modifications to increase the biological activity in a polypeptide having acetylating aldehyde dehydrogenase activity. This can be provided for example by introducing a heterologous nucleic acid molecule encoding a heterologous polypeptide having acetylating aldehyde dehydrogenase activity in the yeast cell. As used in the present disclosure, a polypeptide having acetylating aldehyde dehydrogenase activity has the ability to convert acetyl-coA into an aldehyde. In some embodiments, the polypeptide having acetylating aldehyde dehydrogenase activity is an acetaldehyde/alcohol dehydrogenases (AADH) or is a bifunctional acetylating aldehyde dehydrogenase/alcohol dehydrogenase (ADHE). The bifunctional acetaldehyde/alcohol dehydrogenase is an enzyme capable of converting acetyl-CoA into acetaldehyde as well as acetaldehyde into ethanol. Heterologous bifunctional acetaldehyde/alcohol dehydrogenases include but are not limited to those described in U.S. Pat. Ser. No. 8,956,851 and WO 2015/023989, both incorporated herewith in their entirety. Heterologous AADHs of the present disclosure include, but are not limited to, the ADHE polypeptides or a polypeptide encoded by an adhe gene ortholog. In an embodiment, the AADH is from a Bifidobacterium sp., such as for example, a Bifidobacterium adolescentis. In such embodiment, the genetic modification can comprise introducing a heterologous nucleic acid molecule encoding a polypeptide having acetylating aldehyde dehydrogenase activity in the recombinant yeast cell.

In some embodiments, the yeast cell can also include one or more genetic modifications limiting the production of glycerol. For example, the genetic modification can be a genetic modification leading to the reduction in the production, and in an embodiment to the inhibition in the production, of one or more native enzymes that function to produce glycerol. As used in the context of the present disclosure, the expression “reducing the production of one or more native enzymes that function to produce glycerol” refers to a genetic modification which limits or impedes the expression of genes associated with one or more native polypeptides (in some embodiments enzymes) that function to produce glycerol, when compared to a corresponding yeast strain which does not bear such genetic modification. In some instances, the additional genetic modification reduces but still allows the production of one or more native polypeptides that function to produce glycerol. In other instances, the genetic modification inhibits the production of one or more native enzymes that function to produce glycerol. Polypeptides that function to produce glycerol refer to polypeptides which are endogenously found in the yeast cell. Native enzymes that function to produce glycerol include, but are not limited to, the GPD1 and the GPD2 polypeptide (also referred to as GPD1 and GPD2, respectively) as well as the GPP1 and the GPP2 polypeptides (also referred to as GPP1 and GPP2, respectively). In an embodiment, the yeast cell bears a genetic modification in at least one of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). In another embodiment, the yeast cell bears a genetic modification in at least two of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). Examples of recombinant yeast cells bearing such genetic modification(s) leading to the reduction in the production of one or more native enzymes that function to produce glycerol are described in WO 2012/138942, incorporated herewith in its entirety. In some embodiments, the yeast cell has a genetic modification (such as a genetic deletion or insertion) only in one enzyme that functions to produce glycerol, in the gpd2 gene, which would cause the yeast cell to have a knocked-out gpd2 gene. In some embodiments, the recombinant yeast host cell can have a genetic modification in the gpd1 gene and the gpd2 gene resulting is a recombinant yeast host cell being knock-out for the gpd1 gene and the gpd2 gene. In some specific embodiments, the yeast cell can be a knock-out for the gpd1 gene and have duplicate copies of the gpd2 gene (in some embodiments, under the control of the gpd1 promoter). In yet another embodiment, the yeast cell does not bear such genetic modification and includes its native genes coding for the GPP/GDP proteins. As such, in some embodiments, there are no genetic modifications leading to the reduction in the production of one or more native enzymes that function to produce glycerol in the yeast cell.

Alternatively or in combination, the yeast cell can also include one or more additional genetic modifications facilitating the transport of glycerol in the yeast cell. For example, the additional genetic modification can be a genetic modification leading to the increase in activity of one or more native enzymes that function to transport glycerol. In some embodiments, the additional genetic modification is the introduction of a heterologous polypeptide encoding a glycerol transporter. Native enzymes that function to transport glycerol synthesis include, but are not limited to, the FPS1 polypeptide as well as the STL1 polypeptide. The FPS1 polypeptide is a glycerol exporter and the STL1 polypeptide functions to import glycerol in the recombinant yeast host cell. By either reducing or inhibiting the expression of the FPS1 polypeptide and/or increasing the expression of the STL1 polypeptide, it is possible to control, to some extent, glycerol synthesis.

The STL1 protein is natively expressed in yeasts and fungi, therefore the heterologous protein functioning to import glycerol can be derived from yeasts and fungi. STL1 genes encoding the STL1 protein include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID: 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID: 19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus STL1 and Pichia sorbitophilia. In an embodiment, the STL1 protein is encoded by Saccharomyces cerevisiae Gene ID: 852149.

Alternatively or in combination, the yeast cell can have a genetic modification allowing the expression of a saccharolytic enzyme. For example, the additional genetic modification can be a genetic modification leading to the increase in expression of one or more native sccharolytic enzyme. In some embodiments, the additional genetic modification is the introduction of a heterologous polypeptide encoding a saccharolytic enzyme. As used in the context of the present disclosure, a “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes. amylolytic enzyme. In an embodiment, the saccharolytic enzyme is an amylolytic enzyme. As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in WO2018/167670 and are incorporated herein by reference.

For example, the yeast cell can bear one or more genetic modifications allowing for the production of a heterologous glucoamylase. Many microbes produce an amylase to degrade extracellular starches. In addition to cleaving the last α(1-4) glycosidic linkages at the non-reducing end of amylose and amylopectin, yielding glucose, γ-amylase will cleave α(1-6) glycosidic linkages. The heterologous glucoamylase can be derived from any organism. In an embodiment, the heterologous protein is derived from a γ-amylase, such as, for example, the glucoamylase of Saccharomycopsis filbuligera (e.g., encoded by the glu 0111 gene). Examples of yeast cells bearing such genetic modifications are described in WO 2011/153516 as well as in WO 2017/037614 and herewith incorporated in their entirety. In an embodiment, the recombinant yeast host cell is capable of expressing the heterologous glucoamylase having the amino acid sequence of SEQ ID NO: 28, a variant of the amino acid sequence of SEQ ID NO: 28 having glucoamylase activity or is a fragment of the amino acid sequence of SEQ ID NO: 28 having glucoamylase activity. In some embodiments, the heterologous nucleic acid molecule encoding the polypeptide having glucoamylase activity has the nucleic acid sequence of SEQ ID NO: 29, is a variant of the nucleic acid sequence of SEQ ID NO: 29 (encoding a polypeptide having glucoamylase activity), is a fragment of the nucleic acid sequence of SEQ ID NO: 29 (encoding a polypeptide having glucoamylase activity) or is a degenerate nucleic acid sequence encoding the polypeptide of SEQ ID NO: 28 (its variants or its fragments).

Alternatively or in combination, the yeast cell can have increased biological activity in one or more involved in formate/acetyl-CoA production polypeptide. For example, the recombinant yeast host cell can bear one or more genetic modifications for increasing formate/acetyl-CoA production. In order to do so, yeast cell can bear one or more genetic modification for increasing its pyruvate formate lyase activity. For example, the yeast cell can have one or more heterologous nucleic acid molecules encoding one or more polypeptide having formate lyase activity. As used in the context of the present disclosure, “a heterologous enzyme that function to increase formate/acetyl-CoA production” refers to polypeptides which may or may not be endogenously found in the recombinant yeast host cell and that are purposefully introduced into the yeast cells to anabolize formate. In some embodiments, the heterologous enzyme that can be a heterologous pyruvate formate lyase (PFL), such as PFLA or PFLB heterologous PFL of the present disclosure include, but are not limited to, the PFLA polypeptide, a polypeptide encoded by a pfla gene ortholog, the PFLB polypeptide or a polypeptide encoded by a pflb gene ortholog.

Embodiments of the pyruvate formate lyase activating enzyme and of PFLA can be derived, without limitation, from the following (the number in brackets correspond to the Gene ID number): Escherichia coli (MG1655945517), Shewanella oneidensis (1706020), Bifidobacterium longum (1022452), Mycobacterium bovis (32287203), Haemophilus parasuis (7277998), Mannheimia haemolytica (15341817), Vibrio vulnificus (33955434), Cronobacter sakazakii (29456271), Vibrio alginolyticus (31649536), Pasteurella multocida (29388611), Aggregatibacter actinomycetemcomitans (31673701), Actinobacillus suis (34291363), Finegoldia magna (34165045), Zymomonas mobilis subsp. mobilis (3073423), Vibrio tubiashfi (23444968), Gallibacterium anatis (10563639), Actinobacillus pleuropneumoniae serovar (4849949), Ruminiclostridium thermocellum (35805539), Cylindrospermopsis raciborskii (34474378), Lactococcus garvieae (34204939), Bacillus cytotoxicus (33895780), Providencia stuartii (31518098), Pantoea ananatis (31510290), Teredinibacter turnerae (29648846), Morganella morganii subsp. morganii (14670737), Vibrio anguillarum (77510775106), Dickeya dadantii (39379733484), Xenorhabdus bovienii (8830449), Edwardsiella ictaluri (7959196), Proteus mirabilis (6801040), Rahnella aquatilis (34350771), Bacillus pseudomycoides (34214771), Vibrio alginolyticus (29867350), Vibrio nigripulchritudo (29462895), Vibrio orientalis (25689084), Kosakonia sacchari (23844195), Serratia marcescens subsp. marcescens (23387394), Shewanella baltica (11772864), Vibrio vulnificus (2625152), Streptomyces acidiscabies (33082227), Streptomyces davaonensis (31227069), Streptomyces scabiei (24308152), Volvox carteri f. nagariensis (9616877), Vibrio breoganii (35839746), Vibrio mediterranei (34766273), Fibrobacter succinogenes subsp. succinogenes (34755395), Enterococcus gilvus (34360882), Akkermansia muciniphila (34173806), Enterobacter hormaechei subsp. Steigerwaltii (34153767), Dickeya zeae (33924935), Enterobacter sp. (32442159), Serratia odorifera (31794665), Vibrio crassostreae (31641425), Selenomonas ruminantium subsp. lactilytica (31522409), Fusobacterium necrophorum subsp. funduliforme (31520833), Bacteroides uniformis (31507008), Haemophilus somnus (233631487328), Rodentibacter pneumotropicus (31211548), Pectobacterium carotovorum subsp. carotovorum (29706463), Eikenella corrodens (29689753), Bacillus thuringiensis (29685036), Streptomyces rimosus subsp. Rimosus (29531909), Vibrio fluvialis (29387180), Klebsiella oxytoca (29377541), Parageobacillus thermoglucosidans (29237437), Aeromonas veronii (28678409), Clostridium innocuum (26150741), Neisseria mucosa (25047077), Citrobacter freundii (23337507), Clostridium bolteae (23114831), Vibrio tasmaniensis (7160642), Aeromonas salmonicida subsp. salmonicida (4995006), Escherichia coli O157:H7 str. Sakai (917728), Escherichia coli O83:H1 str. (12877392), Yersinia pestis (11742220), Clostridioides difficile (4915332), Vibrio fischeri (3278678), Vibrio parahaemolyticus (1188496), Vibrio corallfilyticus (29561946), Kosakonia cowanii (35808238), Yersinia ruckeri (29469535), Gardnerella vaginalis (99041930), Listeria fleischmannii subsp. Coloradonensis (34329629), Photobacterium kishitanii (31588205), Aggregatibacter actinomycetemcomitans (29932581), Bacteroides caccae (36116123), Vibrio toranzoniae (34373279), Providencia alcalifaciens (34346411), Edwardsiella anguillarum (33937991), Lonsdalea quercina subsp. Quercina (33074607), Pantoea septica (32455521), Butyrivibrio proteoclasticus (31781353), Photorhabdus temperata subsp. Thracensis (29598129), Dickeya solani (23246485), Aeromonas hydrophila subsp. hydrophila (4489195), Vibrio cholerae O1 biovar El Tor str. (2613623), Serratia rubidaea (32372861), Vibrio bivalvicida (32079218), Serratia liquefaciens (29904481), Gilliamella apicola (29851437), Pluralibacter gergoviae (29488654), Escherichia coli O104:H4 (13701423), Enterobacter aerogenes (10793245), Escherichia coli (7152373), Vibrio campbellii (5555486), Shigella dysenteriae (3795967), Bacillus thuringiensis serovar konkukian (2854507), Salmonella enterica subsp. enterica serovar Typhimurium (1252488), Bacillus anthracis (1087733), Shigella flexneri (1023839), Streptomyces griseoruber (32320335), Ruminococcus gnavus (35895414), Aeromonas fluvialis (35843699), Streptomyces ossamyceticus (35815915), Xenorhabdus doucetiae (34866557), Lactococcus piscium (34864314), Bacillus glycinifermentans (34773640), Photobacterium damselae subsp. Damselae 34509297, Streptomyces venezuelae 34035779, Shewanella algae (34011413), Neisseria sicca (33952518), Chania multitudinisentens (32575347), Kitasatospora purpeofusca (32375714), Serratia fonticola (32345867), Aeromonas enteropelogenes (32325051), Micromonospora aurantiaca (32162988), Moritella viscosa (31933483), Yersinia aldovae (31912331), Leclercia adecarboxylata (31868528), Salinivibrio costicola subsp. costicola (31850688), Aggregatibacter aphrophilus (31611082), Photobacterium leiognathi (31590325), Streptomyces canus (31293262), Pantoea dispersa (29923491), Pantoea rwandensis (29806428), Paenibacillus borealis (29548601), Aliivibrio wodanis (28541257), Streptomyces virginiae (23221817), Escherichia coli (7158493), Mycobacterium tuberculosis (887973), Streptococcus mutans (1028925), Streptococcus cristatus (29901602), Enterococcus hirae (13176624), Bacillus licheniformis (3031413), Chromobacterium violaceum (24949178), Parabacteroides distasonis (5308542), Bacteroides vulgatus (5303840), Faecalibacterium prausnitzii (34753201), Melissococcus plutonius (34410474), Streptococcus gallolyticus subsp. gallolyticus (34397064), Enterococcus malodoratus (34355146), Bacteroides oleiciplenus (32503668), Listeria monocytogenes (985766), Enterococcus faecalis (1200510), Campylobacter jejuni subsp. jejuni (905864), Lactobacillus plantarum (1063963), Yersinia enterocolitica subsp. enterocolitica (4713333), Streptococcus equinus (33961143), Macrococcus canis (35294771), Streptococcus sanguinis (4807186), Lactobacillus salivarius (3978441), Lactococcus lactis subsp. lactis (1115478), Enterococcus faecium (12999835), Clostridium botulinum A (5184387), Clostridium acetobutylicum (1117164), Bacillus thuringiensis serovar konkukian (2857050), Cryobacterium flavum (35899117), Enterovibrio norvegicus (35871749), Bacillus acidiceler (34874556), Prevotella intermedia (34516987), Pseudobutyrivibrio ruminis (34419801), Pseudovibrio ascidiaceicola (34149433), Corynebacterium coyleae (34026109), Lactobacillus curvatus (33994172), Cellulosimicrobium cellulans (33980622), Lactobacillus agilis (33975995), Lactobacillus sakei (33973512), Staphylococcus simulans (32051953), Obesumbacterium proteus (29501324), Salmonella enterica subsp. enterica serovar Typhi (1247402), Streptococcus agalactiae (1014207), Streptococcus agalactiae (1013114), Legionella pneumophila subsp. pneumophila str. Philadelphia (119832735), Pyrococcus furiosus (1468475), Mannheimia haemolytica (15340992), Thalassiosira pseudonana (7444511), Thalassiosira pseudonana (7444510), Streptococcus thermophilus (31940129), Sulfolobus solfataricus (1454925), Streptococcus iniae (35765828), Streptococcus iniae (35764800), Bifidobacterium thermophilum (31839084), Bifidobacterium animalis subsp. lactis (29695452), Streptobacillus moniliformis (29673299), Thermogladius calderae (13013001), Streptococcus oralis subsp. tigurinus (31538096), Lactobacillus ruminis (29802671), Streptococcus parauberis (29752557), Bacteroides ovatus (29454036), Streptococcus gordonii str. Challis substr. CH1 (25052319), Clostridium botulinum B str. Eklund 17B (19963260), Thermococcus litoralis (16548368), Archaeoglobus sulfaticallidus (15392443), Ferroglobus placidus (8778929), Archaeoglobus profundus (8739370), Listeria seeligeri serovar 1/2b (32488230), Bacillus thuringiensis (31632063), Rhodobacter capsulatus (31491679), Clostridium botulinum (29749009), Clostridium perfringens (29571530), Lactococcus garvieae (12478921), Proteus mirabilis (6799920), Lactobacillus animalis (32012274), Vibrio alginolyticus (29869205), Bacteroides thetaiotaomicron (31617701), Bacteroides thetaiotaomicron (31617140), Bacteroides cellulosilyticus (29608790), Bacteroides ovatus (29453452), Bacillus mycoides (29402181), Chlamydomonas reinhardtii (5726206), Fusobacterium periodonticum (35833538), Selenomonas flueggei (32477557), Selenomonas noxia (32475880), Anaerococcus hydrogenalis (32462628), Centipeda periodontii (32173931), Centipeda periodontii (32173899), Streptococcus thermophilus (31938326), Enterococcus durans (31916360), Fusobacterium nucleatum (31730399), Anaerostipes hadrus (31625694), Anaerostipes hadrus (31623667), Enterococcus haemoperoxidus (29838940), Gardnerella vaginalis(29692621), Streptococcus salivarius (29397526), Klebsiella oxytoca (29379245), Bifidobacterium breve (29241363), Actinomyces odontolyticus (25045153), Haemophilus ducreyi (24944624), Archaeoglobus fulgidus (24793671), Streptococcus uberis (24161511), Fusobacterium nucleatum subsp. animalis (23369066), Corynebacterium accolens (23249616), Archaeoglobus veneficus (10394332), Prevotella melaninogenica (9497682), Aeromonas salmonicida subsp. salmonicida (4997325), Pyrobaculum islandicum (4616932), Thermofilum pendens (4600420), Bifidobacterium adolescentis (4556560), Listeria monocytogenes (986485), Bifidobacterium thermophilum (35776852), Methanothermobacter sp. CaT2 (24854111), Streptococcus pyogenes (901706), Exiguobacterium sibiricum (31768748), Clostridioides difficile (4916015), Clostridioides difficile (4913022), Vibrio parahaemolyticus (1192264), Yersinia enterocolitica subsp. enterocolitica (4712948), Enterococcus cecorum (29475065), Bifidobacterium pseudolongum (34879480), Methanothermus fervidus (9962832), Methanothermus fervidus (9962056), Corynebacterium simulans (29536891), Thermoproteus uzoniensis (10359872),Vulcanisaeta distributa (9752274), Streptococcus mitis (8799048), Ferroglobus placidus (8778420), Streptococcus suis (8153745), Clostridium novyi (4541619), Streptococcus mutans (1029528), Thermosynechococcus elongatus (1010568), Chlorobium tepidum (1007539), Fusobacterium nucleatum subsp. nucleatum (993139), Streptococcus pneumoniae (933787), Clostridium baratii (31579258), Enterococcus mundtii (31547246), Prevotella ruminicola (31500814), Aeromonas hydrophila subsp. hydrophila (4490168), Aeromonas hydrophila subsp. hydrophila (4487541), Clostridium acetobutylicum (1117604), Chromobacterium subtsugae (31604683), Gilliamella apicola (29849369), Klebsiella pneumoniae subsp. pneumoniae (11846825), Enterobacter cloacae subsp. cloacae (9125235), Escherichia coli (7150298), Salmonella enterica subsp. enterica serovar Typhimurium (1252363), Salmonella enterica subsp. enterica serovar Typhi (1247322), Bacillus cereus (1202845), Bacteroides thetaiotaomicron (1074343), Bacteroides thetaiotaomicron (1071815), Bacillus coagulans (29814250), Bacteroides cellulosilyticus (29610027), Bacillus anthracis (2850719), Monoraphidium neglectum (25735215), Monoraphidium neglectum (25727595), Alloscardovia omnicolens (35868062), Actinomyces neuii subsp. neuii (35867196), Acetoanaerobium sticklandii (35557713), Exiguobacterium undae (32084128), Paenibacillus pabuli (32034589), Paenibacillus etheri (32019864), Actinomyces oris (31655321), Vibrio alginolyticus (31651465), Brochothrix thermosphacta (29820407), Lactobacillus sakei subsp. sakei (29638315), Anoxybacillus gonensis (29574914), variants thereof as well as fragments thereof. In an embodiment, the PFLA protein is derived from the genus Bifidobacterium and in some embodiments from the species Bifidobacterium adolescentis.

Embodiments of PFLB can be derived, without limitation, from the following (the number in brackets correspond to the Gene ID number): Escherichia coli (945514), Shewanella oneidensis (1170601), Actinobacillus suis (34292499), Finegoldia magna (34165044), Streptococcus cristatus (29901775), Enterococcus hirae (13176625), Bacillus (3031414), Providencia alcalifaciens (34345353), Lactococcus garvieae (34203444), Butyrivibrio proteoclasticus (31781354), Teredinibacter turnerae (29651613), Chromobacterium violaceum (24945652), Vibrio campbellii (5554880), Vibrio campbellii (5554796), Rahnella aquatilis HX2 (34351700), Serratia rubidaea (32375076), Kosakonia sacchari SP1 (23845740), Shewanella baltica (11772863), Streptomyces acidiscabies (33082309), Streptomyces davaonensis (31227068), Parabacteroides distasonis (5308541), Bacteroides vulgatus (5303841), Fibrobacter succinogenes subsp. succinogenes (34755392), Photobacterium damselae subsp. Damselae (34512678), Enterococcus gilvus (34361749), Enterococcus gilvus (34360863), Enterococcus malodoratus (34355213), Enterococcus malodoratus (34354022), Akkermansia muciniphila (34174913), Lactobacillus curvatus (33995135), Dickeya zeae (33924934), Bacteroides oleiciplenus (32502326), Micromonospora aurantiaca (32162989), Selenomonas ruminantium subsp. lactilytica (31522408), Fusobacterium necrophorum subsp. funduliforme (31520832), Bacteroides uniformis (31507007), Streptomyces rimosus subsp. Rimosus (29531908), Clostridium innocuum (26150740), Haemophilus] ducreyi (24944556), Clostridium bolteae (23114829), Vibrio tasmaniensis (7160644), Aeromonas salmonicida subsp. salmonicida (4997718), Listeria monocytogenes (986171), Enterococcus faecalis (1200511), Lactobacillus plantarum (1064019), Vibrio fischeri (3278780), Lactobacillus sakei (33973511), Gardnerella vaginalis (9904192), Vibrio vulnificus (33954428), Vibrio toranzoniae (34373229), Anaerostipes hadrus (34240161), Edwardsiella anguillarum (33940299), Edwardsiella anguillarum (33937990), Lonsdalea quercina subsp. Quercina (33074710), Enterococcus faecium (12999834), Aeromonas hydrophila subsp. hydrophila (4489100), Clostridium acetobutylicum (1117163), Escherichia coli (7151395), Shigella dysenteriae (3795966), Bacillus thuringiensis serovar konkukian (2856201), Salmonella enterica subsp. enterica serovar Typhimurium (1252491), Shigella flexneri (1023824), Streptomyces griseoruber (32320336), Cryobacterium flavum (35898977), Ruminococcus gnavus (35895748), Bacillus acidiceler (34874555), Lactococcus piscium (34864362), Vibrio mediterranei (34766270), Faecalibacterium prausnitzii (34753200), Prevotella intermedia (34516966), Photobacterium damselae subsp. Damselae (34509286), Pseudobutyrivibrio ruminis (34419894), Melissococcus plutonius (34408953), Streptococcus gallolyticus subsp. gallolyticus (34398704), Enterobacter hormaechei subsp. Steigerwaltii (34155981), Enterobacter hormaechei subsp. Steigerwaltii (34152298), Streptomyces venezuelae (34036549), Shewanella algae (34009243), Lactobacillus agilis (33976013), Streptococcus equinus (33961013), Neisseria sicca (33952517),Kitasatospora purpeofusca (32375782), Paenibacillus borealis (29549449), Vibrio fluvialis (29387150), Aliivibrio wodanis (28542465), Aliivibrio wodanis (28541256), Escherichia coli (7157421), Salmonella enterica subsp. enterica serovar Typhi (1247405), Yersinia pestis (1174224), Yersinia enterocolitica subsp. enterocolitica (4713334), Streptococcus suis (8155093), Escherichia coli (947854), Escherichia coli (946315), Escherichia coli (945513), Escherichia coli (948904) , Escherichia coli (917731), Yersinia enterocolitica subsp. enterocolitica (4714349), variants thereof as well as fragments thereof. In an embodiment, the PFLB protein is derived from the genus Bifidobacterium and in some embodiments from the specifies Bifidobacterium adolescentis.

In some embodiments, the yeast cell comprises a genetic modification for expressing a PFLA protein, a PFLB protein or a combination. In a specific embodiment, the yeast cell comprises a genetic modification for expressing a PFLA protein and a PFLB protein which can, in some embodiments, be provided on distinct heterologous nucleic acid molecules. As indicated below, the recombinant yeast host cell can also include additional genetic modifications to provide or increase its ability to transform acetyl-CoA into an alcohol such as ethanol.

Alternatively or in combination, the yeast cell can have increased biological activity in a polypeptide capable of utilizing acetyl-CoA. For example, the yeast cell can bear one or more genetic modifications for utilizing acetyl-CoA for example, by providing or increasing acetaldehyde and/or alcohol dehydrogenase activity. For example, the yeast cell can have one or more heterologous nucleic acid molecules encoding one or more heterologous polypeptide for utilizing acetyl-CoA. Acetyl-CoA can be converted to an alcohol such as ethanol using second an acetaldehyde dehydrogenase and then an alcohol dehydrogenase. Acylating acetaldehyde dehydrogenases (E.C. 1.2.1.10) are known to catalyze the conversion of acetaldehyde into acetyl-CoA in the presence of CoA. Alcohol dehydrogenases (E.C. 1.1.1.1) are known to be able to catalyze the conversion of acetaldehyde into ethanol. The acetaldehyde dehydrogenase and alcohol dehydrogenase activity can be provided by a single protein (e.g., a bifunctional acetaldehyde/alcohol dehydrogenase) or by a combination of more than one protein (e.g., an acetaldehyde dehydrogenase and an alcohol dehydrogenase). In embodiments in which the acetaldehyde/alcohol dehydrogenase activity is provided by more than one protein, it may not be necessary to provide the combination of proteins in a recombinant form in the recombinant yeast host cell as the cell may have some pre-existing acetaldehyde or alcohol dehydrogenase activity. In such embodiments, the genetic modification can include providing one or more heterologous nucleic acid molecule encoding one or more of a heterologous acetaldehyde dehydrogenase (AADH), a heterologous alcohol dehydrogenase (ADH) and/or heterologous bifunctional acetaldehyde/alcohol dehydrogenases (ADHE). In another embodiment, the genetic modification comprises introducing a heterologous nucleic acid encoding a heterologous bifunctional acetaldehyde/alcohol dehydrogenases (AADH) such as those described in U.S. Pat. Ser. No. 8,956,851 and WO 2015/023989, both incorporated herewith in their entirety. Heterologous AADHs of the present disclosure include, but are not limited to, the ADHE polypeptides or a polypeptide encoded by an adhe gene ortholog.

The recombinant LAB host cell described herein can be provided as a combination with the yeast cell described herein. In such combination, the recombinant can be provided in a distinct container from the yeast cell. The recombinant LAB host cell can be provided as a cell concentrate. The cell concentrate comprising the recombinant LAB host cell can be obtained, for example, by propagating the recombinant LAB host cell in a culture medium and removing at least one components of the medium comprising the propagated recombinant LAB host cells. This can be done, for example, by dehydrating, filtering (including ultra-filtrating) and/or centrifuging the medium comprising the propagated recombinant LAB host cells. In an embodiment, the recombinant LAB can be provided as a frozen concentrate in the combination. The yeast cell can be provided as a cell concentrate. The cell concentrate comprising the yeast cell can be obtained, for example, by propagating the yeast cells in a culture medium and removing at least one components of the medium comprising the propagated yeast host cell. This can be done, for example, by dehydrating, filtering (including ultra-filtrating) and/or centrifuging the medium comprising the propagated yeast cells. In an embodiment, the yeast cell is provided as a cream in the combination.

### Distiller's Product and Associated Feed Products

The present disclosure provides a whole stillage having a modulated nutritional content. Since distillers products are made from the whole stillage, the distillers products of the present disclosure will also have a modulated nutritional content. The nutritional content of the whole stillage/distillers products are “modulated” because they differ from the nutritional content of control whole stillage/distillers products which could have been obtained under the same conditions but in the absence of the recombinant LAB host cell. The differences between the whole stillage/distillers products of the present disclosure and those obtained from a control fermentation in the absence of the recombinant LAB host cell can be observed in the protein content, the amino acid profile, the fiber content, the lipid content and/or the starch content. In one embodiment, the whole stillage/distillers products of the present disclosure has an increase in protein content when compared to the whole stillage/distillers product obtained from a control fermentation in the absence of the recombinant LAB host cell. In a specific embodiment, the whole stillage/distillers products of the present disclosure has an increase of at least 0.1, 0.2, 0.3, 0.4, 0.5, 0.6, 0.7, 0.8, 0.9, 1.0, 1.1, 1.2, 1.3, 1.4, 1.5, 1.6, 1.7% w/w or higher protein content when compared to a control stilage stillage/distillers product obtained from a control fermentation in the absence of the recombinant LAB host cell. In some embodiments, the difference in the protein content between the whole stillage/distillers products of the present disclosure and the whole stillage/control distillers product is statistically significant. In one embodiment, the whole stillage/distillers products of the present disclosure has an increase in fiber content (total fiber, crude fiber, neutral detergent fiber and/or acid detergent fiber) when compared to the whole stillage/distillers product obtained from a control fermentation in the absence of the recombinant LAB host cell. In a embodiment, the whole stillage/distillers products of the present disclosure has an increase in the total fiber content of at least 0.5, 1.0, 1.5, 2.0, 2.5, 3.0, 3.5, 4.0, 4.5, 4.8% w/w or higher when compared to the whole stillage/distillers product obtained from a control fermentation in the absence of the recombinant LAB host cell. In a embodiment, the whole stillage/distillers products of the present disclosure has an increase in the crude fiber content of at least 0.5, 1.0, 1.5, 2.0, 2.5, 3.0, 3.5, 4.0, 4.5, 4.8% w/w or higher when compared to the whole stillage/distillers product obtained from a control fermentation in the absence of the recombinant LAB host cell. In a embodiment, the whole stillage/distillers products of the present disclosure has an increase in the neutral fiber content of at least 0.5, 1.0, 1.5, 2.0, 2.5, 3.0, 3.5, 4.0% w/w or higher when compared to the whole stillage/distillers product obtained from a control fermentation in the absence of the recombinant LAB host cell. In a embodiment, the whole stillage/distillers products of the present disclosure has an increase in the acid detergent content of at least 0.5, 1.0, 1.5, 2.0, 2.5, 3.0, 3.5, 4.0, 4.5, 5.0, 5.5, 6.0, 6.5, 7.0% w/w or higher when compared to the whole stillage/distillers product obtained from a control fermentation in the absence of the recombinant LAB host cell. In one embodiment, the whole stillage/distillers products of the present disclosure has an increase in crude fat content when compared to the whole stillage/distillers product obtained from a control fermentation in the absence of the recombinant LAB host cell. In an embodiment, the whole stillage/distillers products of the present disclosure has an increase in crude fat content of at least 0.1, 0.2, 0.3, 0.4, 0.5, 0.6, 0.7, 0.8, 0.9, 1.0% w/w or higher when compared to the whole stillage/distillers product obtained from a control fermentation in the absence of the recombinant LAB host cell. In one embodiment, the whole stillage/distilled products of the present disclosure has a decrease in starch content when compared to the whole stillage/distillers product obtained from a control fermentation in the absence of the recombinant LAB host cell. In an embodiment, the whole stillage/distilled products of the present disclosure has a decrease in starch content of at least 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 16.8% w/w or higher when compared to the whole stillage/distillers product obtained from a control fermentation in the absence of the recombinant LAB host cell.

In some embodiments, the whole stillage/distillers products of the present disclosure can have a different amino acid profile than the control whole stillage/distillers products obtained in a fermentation that did not include the recombinant LAB host cell. In an embodiment, the content in alanine in the whole stillage/distillers product is increased with respect to the control whole stillage/distillers products. In a further embodiment, the content in alanine in the whole stillage/distillers product is increased in a statistically significant manner with respect to the control whole stillage/distillers products. In an embodiment, the content in arginine in the whole stillage/distillers product remains substantially the same or is increased with respect to the control whole stillage/distillers products. In an embodiment, the content in asparagine in the whole stillage/distillers product is increased with respect to the control whole stillage/distillers products. In an embodiment, the content in aspartic acid in the whole stillage/distillers product is increased with respect to the control whole stillage/distillers products. In an embodiment, the content in asparagine and in aspartic acid in the whole stillage/distillers product is increased with respect to the control whole stillage/distillers products. In a further embodiment, the content in asparagine and in aspartic acid in the whole stillage/distillers product is increased in a statistically significant manner with respect to the control whole stillage/distillers products. In an embodiment, the content in glutamic acid and in glutamine in the whole stillage/distillers product is increased with respect to the control whole stillage/distillers products. In another embodiment, the content in glutamic acid and in glutamine in the whole stillage/distillers product is increased in a statistically significant manner with respect to the control whole stillage/distillers products. In an embodiment, the content in glycine in the whole stillage/distillers product remains substantially the same or is increased with respect to the control whole stillage/distillers products. In an embodiment, the content in histidine in the whole stillage/distillers product remains substantially the same or is increased with respect to the control whole/stillage distillers products. In an embodiment, the content in histidine in the whole stillage/distillers product remains substantially the same or is increased with respect to the control whole stillage/distillers products. In an embodiment, the content in leucine in the whole stillage/distillers product is increased with respect to the control whole stillage/distillers products. In a further embodiment, the content in leucine in the whole stillage/distillers product is increased in a statistically significant manner with respect to the control whole stillage/distillers products. In an embodiment, the content in phenylalanine in the whole stillage/distillers product is increased with respect to the control whole stillage/distillers products. In a further embodiment, the content in phenylalanine in the whole stillage/distillers product is increased in a statistically significant manner with respect to the control whole stillage/distillers products. In an embodiment, the content in proline in the whole stillage/distillers product remains substantially the same or is decreased with respect to the control whole stillage/distillers products. In an embodiment, the content in serine in the whole stillage/distillers product is increased with respect to the control whole stillage/distillers products. In an embodiment, the content in threonine in the whole stillage/distillers product remains substantially the same or is decreased with respect to the control whole stillage/distillers products. In an embodiment, the content in lysine in the whole stillage/distillers product remains substantially the same or is decreased with respect to the control whole stillage/distillers products. In an embodiment, the content in tyrosine in the whole stillage/distillers product is increased with respect to the control whole stillage/distillers products. In an embodiment, the content in valine in the whole stillage/distillers product is increased with respect to the control whole stillage/distillers products. In an embodiment, the content in cysteine in the whole stillage/distillers product remains substantially the same or is increased with respect to the control whole stillage/distillers products. In an embodiment, the content in methionine in the whole stillage/distillers product remains substantially the same or is increased with respect to the control whole stillage/distillers products. In an embodiment, the content in tryptophan in the whole stillage/distillers product remains substantially the same or is increased with respect to the control whole stillage/distillers products. In an embodiment, the content in alanine, aspartic acid, asparagine, glutamine, glutamic acid, leucine and phenylalanine in the whole stillage/distillers product is increased with respect to the control whole stillage/distillers products (and this increase can be, in some embodiments, a statistically significant increase). In an embodiment, the content in leucine and phenylalanine in the whole stillage/distillers product is increased with respect to the control whole stillage/distillers products (and this increase can be, in some embodiments, a statistically significant increase).

In embodiments in which the recombinant LAB host cell is capable of making gamma-aminobutyric acid and as such, the whole stillage/distillers products will also include gamma-aminobutyric acid.

The distillers product of the present disclosure is obtainable or obtained by submitting a biomass to a fermentation with the recombinant LAB host cell and the fermenting yeast and deriving one or more by-product from the whole stillage obtained. Thus, the whole stillage and the distillers products of the present disclosure comprises one or more component of the recombinant LAB host cell that was present during the fermentation step. Components of the recombinant LAB host cell susceptible to be present in the whole stillage/distillers product of the present disclosure include, but are not limited to, a cell membrane or a component derived from the cell membrane (e.g., a pilus or a component of a pilus, a flagella or a component of a flagella, a capsule or a component of a capsule, a cell wall or a component of a cell wall, a plasma membrane or a component of a plasma membrane (a membrane protein for example), an organelle or a component derived from an organelle, a cytoskeleton or a component of a cytoskeleton, an inclusion or a component of an inclusion, a vacuole or a component of a vacuole, a nucleus or a component derived from a nucleus (a deoxyribonucleic acid for example). The bacterial component can include one or more amino acid residues, one or more lipid and/or one or more carbohydrate.

The distillers product of the present disclosure can be distillers wet grains (DWG). The DWG corresponds to the solid fraction of the whole stillage (which can also be referred to as a wet cake). The DWG can be obtained by separating the solid fraction of the whole stillage from its liquid fraction (referred to as thin stillage). This can be done, for example, by centrifuging the whole stillage and removing the liquid fraction (e.g., the supernatant or thin stillage) from the solid fraction (located in the pellet). The DWG can be used directly as a feed. Alternatively, it can serve as a feed supplement intended to be admixed with other feed components.

The distillers product of the present disclosure can be distillers dried grains (DDG). The DDG corresponds to the solid fraction of the whole stillage (which can also be referred to as a wet cake) which has been subsequently dried. The DDG can be obtained by separating the solid fraction of the whole stillage (referred to as the wet cake) from its liquid fraction (referred to as the syrup). This can be done, for example, by centrifuging the whole stillage, removing the liquid fraction (e.g., the supernatant or thin stillage) from the solid fraction (located in the pellet, referred to as the wet cake) and drying the solid fraction obtained into the DDG. The DDG can be used directly as a feed. Alternatively, it can serve as a feed supplement intended to be admixed with other feed components.

The distillers product of the present disclosure can be the syrup. The syrup corresponds to the evaported liquid fraction of the whole stillage. The syrup can be obtained by separating the solid fraction of the whole stillage from its liquid fraction. This can be done, for example, by centrifuging the whole stillage, removing the solid fraction from the liquid fraction. The liquid fraction can be evaporated (to reach a percentage of solids between 25 and 55% for example). The syrup can be used directly as a feed. Alternatively, it can serve as a feed supplement intended to be admixed with other feed components.

The wet cake (e.g., solid fraction of the whole stillage) can be supplemented with a syrup to obtain, in a wet form, distillers wet grains with solubles (DWGS) or, in a dried form, distillers dried grains with solubles (DDGS). The syrup can be obtained by evaporating thin stillage. The syrup can be added to the wet cake to obtain the DWGS. Alternatively, the syrup can be added to the wet cake which can be subsequently dried to obtain the DDGS.

The distillers product of the present disclosure can be dried solubles (DS). DS corresponds to the dried syrup. The syrup can be obtained by separating the solid fraction of the whole stillage from its liquid fraction. This can be done, for example, by centrifuging the whole stillage, removing the solid fraction from the liquid fraction. The liquid fraction can be evaporated (to reach a percentage of solids between 25 and 55% for example). The syrup can be then be dried to a solid form (poweder). DS can be used directly as a feed. Alternatively, it can served as a feed supplement intended to be admixed with other feed components.

### EXAMPLE

Lactobacillus paracasei strain 12A was converted to an ethanologen through deletion of four native lactate dehydrogenases, two native mannitol dehydrogenases, and incorporation of a heterologous Production of Ethanol cassette (PET) consisting of the Zymomonas mobilis pyruvate decarboxylase (SEQ ID NO: 1 encoded by SEQ ID NO: 3) and alcohol dehydrogenase (SEQ ID NO: 4 encoded by SEQ ID NO: 6) (ΔL-Idh1::Ppgm-PET, ΔL-Idh2, ΔD-hic, ΔmtID1, ΔmtID2, ΔL-Idh3PuspA-PET). The expression of one PET cassette (including one copy of the Zymomonas mobilis pyruvate decarboxylase and alcohol dehydrogenase) was controlled by the native universal stress protein promoter (uspA) which favors expression during late growth stages. The expression of the other PET cassette (including one copy of the Zymomonas mobilis pyruvate decarboxylase and alcohol dehydrogenase) was controlled by the native phosphoglycerate mutase (pgm) constitutive promoter.

Saccharomyces cerevisiae strain M4080. Strain M4080 expresses a heterologous glucoamylase (SEQ ID NO: 28, encoded by the nucleic acid molecule having the nucleic acid sequence of SEQ ID NO: 30). In some specific embodiments, the recombinant yeast host cell of the present disclosure can express a heterologous nucleic acid molecule comprising the nucleic acid sequence of SEQ ID NO: 30, be a variant of the nucleic acid sequence of SEQ ID NO: 30 (encoding a polypeptide having glucoamylase activity), be a fragment of the nucleic acid sequence of SEQ ID NO: 30 (encoding a polypeptide having glucoamylase activity) or be a degenerate nucleic acid sequence encoding the polypeptide of SEQ ID NO: 28 (its variants or its fragments).

Corn mash fermentation. The fermentation was conducted in a mixture comprising 34% total solids, a 60% glucoamylase dose, 300 ppm of urea and 3 ppm of virginiamycin. It was conducted at a temperature between 30-33° C. for a period of at least 50 hours.

Labscale distillers' dried grain (DDG) production and characterization. After the fermentation was completed, the solids (e.g., wet cake) were harvested by centrifugation (5000 g for 5 minutes). The supernatant was discarded and the pellet was placed into a fluid bed drier. The pellet was first dried for 20 minutes at 65° C. and 100% fan speed. Afterwards, the chunks were broken up and pass through a fines filter, before being dried for for another 20 minutes at 85° C. at 75% fan speed. Afterwards, the chunks were broken up and further dried for 20 minutes at 100° C. at 50% fan speed to obtain the DDG. DDG nutritional content was determined using AOAC reference methods 990.03, 992.15, 982.30, 954.02, 962.09, 994.12, 996.11 and 988.15.

The DDG obtained from the fermentation of a corn mash using S. cerevisiae strain M4080 alone or in combination with Lb. paracasei E5 were characterized. As shown in Table 1, DDG produced from fermentations with a combination of S. cerevisiae and L. paracasei E5 contained less residual starch and significantly (P<0.05) more crude protein than DDG made with the yeast alone. The concentrations of crude fiber, acid detergent fiber, and neutral detergent fiber were also higher in DDG made from the fermentation of a corn mash using both a yeast and Lb. paracasei E5 (Table 1).

TABLE 1
Percent composition of DDG produced using Saccharomyces cerevisiae
M4080 with (M4080 + E5) or without (M4080) Lactobacillus paracasei
E51. Values represent the mean (+standard error) for
DDG samples prepared from six independent fermentations
(three per treatment).

| % w/w | M4080 | ±SE | M4080 + E5 | ±SE | T-Test |
|---|---|---|---|---|---|
| Total Protein | 26.06 | 0.15 | 26.50 | 0.08 | 0.03* |
| Ala | 1.86 | 0.00 | 1.91 | 0.00 | 0.00* |
| Arg | 1.22 | 0.02 | 1.22 | 0.02 | 0.77 |
| Asp/Asn¹ | 1.82 | 0.01 | 1.86 | 0.01 | 0.04* |
| Glu/Gln¹ | 4.21 | 0.00 | 4.33 | 0.02 | 0.01* |
| Gly | 1.15 | 0.02 | 1.15 | 0.00 | 0.69 |
| His | 0.74 | 0.01 | 0.75 | 0.01 | 0.45 |
| Ile | 0.98 | 0.01 | 1.00 | 0.01 | 0.06 |
| Leu | 2.81 | 0.00 | 2.90 | 0.02 | 0.01* |
| Phe | 1.19 | 0.01 | 1.24 | 0.01 | 0.01* |
| Pro | 2.09 | 0.01 | 2.04 | 0.03 | 0.11 |
| Ser | 1.30 | 0.01 | 1.33 | 0.00 | 0.10 |
| Thr | 1.09 | 0.02 | 1.09 | 0.00 | 0.64 |
| Lys | 1.06 | 0.03 | 1.04 | 0.08 | 0.77 |
| Tyr | 0.91 | 0.01 | 0.94 | 0.03 | 0.43 |
| Val | 1.31 | 0.02 | 1.35 | 0.00 | 0.20 |
| Cys | 0.46 | 0.01 | 0.46 | 0.01 | 0.73 |
| Met | 0.52 | 0.01 | 0.53 | 0.00 | 0.39 |
| Trp | 0.25 | 0.00 | 0.26 | 0.01 | 0.39 |
| Starch | 2.80 | 0.70 | 2.33 | 0.76 | 0.56 |
| Total Fiber | 26.43 | 1.04 | 27.70 | 0.93 | 0.27 |
| Crude Fiber | 5.20 | 0.08 | 5.43 | 0.09 | 0.06 |
| Neutral Detergent | 15.07 | 1.03 | 15.67 | 0.17 | 0.50 |
| Fiber | | | | | |
| Acid Detergent Fiber | 6.17 | 0.45 | 6.60 | 1.13 | 0.65 |
| Crude Fat | 18.84 | 0.64 | 18.85 | 0.43 | 0.99 |

²The acid hydrolysis treatment used to generate these data does not allow discrimination between these residues.
*Difference is statistically significant (P < 0.05)

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Fukao M, Obita T, Yoneyama F, Kohda D, Zendo T, Nakayama J, Sonomoto K. Complete covalent structure of nisin Q, new natural nisin variant, containing post-translationally modified amino acids. Biosci Biotechnol Biochem. 2008 July; 72(7):1750-5. O'Connor P M, O'Shea E F, Guinane C M, O'Sullivan O, Cotter P D, Ross R P, Hill C. Nisin H Is a New Nisin Variant Produced by the Gut-Derived Strain Streptococcus hyointestinalis DPC6484. Appl Environ Microbiol. 2015 Jun. 15; 81(12):3953-60. O'Sullivan J N, O'Connor P M, Rea M C, O'Sullivan O, Walsh C J, Healy B, Mathur H, Field D, Hill C, Ross RP. Nisin J, a Novel Natural Nisin Variant, Is Produced by Staphylococcus capitis Sourced from the Human Skin Microbiota. J Bacteriol. 2020 Jan. 15; 202(3). Saunders, J., Rosentrater, K., Krishnan, P. Removal of Color Pigments From Corn Distillers Dried Grains With Solubles (DDGS) to Produce an Upgraded Food Ingredient. J Food Res 2013 Vol. 2, No. 5, 111-123
Wirawan R E, Klesse N A, Jack R W, Tagg J R. Molecular and genetic characterization of a novel nisin variant produced by Streptococcus uberis. Appl Environ Microbiol. 2006 February; 72(2): 1148-56.

What is claimed is:

1. A process of modulating the nutritional content of a whole stillage obtained after the fermentation of a biomass, the process comprising:
(a) contacting a recombinant lactic acid bacteria (LAB) host cell, a yeast and the biomass under conditions to cause the conversion of at least in part of the biomass into a fermentation product and to obtain a fermented biomass comprising the whole stillage and the fermentation product; and
(b) separating the whole stillage from the fermentation product;
wherein the recombinant LAB host cell is capable of expressing one or more first heterologous enzyme for converting the biomass into the fermentation product; and
wherein the whole stillage obtained after step (b) has a different nutritional content than a control whole stillage submitted to step (a) in the absence of the recombinant LAB host cell.

2. The process of claim 1, wherein the whole stillage has, when compared to the control whole stillage:
an increase in protein content;
a different amino acid profile;
an increase in fiber content;
an increase in lipid content; and/or
when the biomass comprises starch, a decrease in starch content.

3. The process of claim 1, wherein the biomass comprises or is obtained from corn and/or the fermentation product comprises or is ethanol.

4. The process of claim 3, wherein the one or more first heterologous enzyme comprises a polypeptide having pyruvate decarboxylase activity and/or a polypeptide having alcohol dehydrogenase activity and the recombinant LAB host cell has a decreased lactate dehydrogenase activity when compared to a corresponding native LAB host cell.

5. The process of claim 1, wherein the biomass comprises one or more bacteriocin and the recombinant LAB host cell expresses (i) one or more second polypeptide conferring immunity to the one or more bacteriocin and/or (ii) the one or more bacteriocin.

6. The process of claim 1, wherein the biomass comprises one or more antibiotic and the recombinant LAB host cell expresses one or more third heterologous polypeptide conferring resistance to the one or more antibiotic or is adapted to be resistant to the antibiotic. The process of claim 1, wherein the recombinant LAB host cell expresses one or more fourth polypeptide having proteolytic activity, wherein the one or more fourth polypeptide is a native polypeptide or a heterologous polypeptide.

8. The process of claim 1, wherein the recombinant LAB host cell expresses one or more fifth polypeptide involved in the metabolism of one or more amino acid, wherein the one or more fifth polypeptide is a native polypeptide or a heterologous polypeptide.

9. The process of claim 8, wherein the one or more amino acid comprises glutamate/gamma-amino butyrate.

10. The process of claim 9, wherein the one or more fifth polypeptide comprises:
a glutamate decarboxylase; and/or
a glutamate/gamma-amino butyrate (GABA) transporter.

11. The process of claim 1, wherein the recombinant LAB host cell is from the genus Lactobacillus sp. and/or from the species Lactobacillus paracasei.

12. The process of claim 1, wherein the yeast is a recombinant yeast host cell, from the genus Saccharomyces sp and/or from the species Saccharomyces cerevisiae.

13. The process of claim 1, comprising, at step (b), distilling the fermented biomass to remove the fermentation product from the whole stillage.

14. The process of claim 1 further comprising centrifuging the whole stillage to separate a thin stillage from a wet cake.

15. The process of claim 14, further comprising:
formulating the wet cake in distillers wet grains (DWG);
drying the wet cake to obtain distillers dried grains (DDG); and/or
evaporating the thin stillage to obtain a syrup.

16. The process of claim 15, further comprising:
adding the syrup to the wet cake to obtain distillers wet grains with solubles (DWGS);
drying the syrup to obtain dried solubles (DS); and/or
further comprising drying the DWGS to obtain distillers dried grains with solubles (DDGS).

17. A composition comprising (i) whole stillage obtainable or obtained by the process of claim 1 and (ii) a component of the recombinant LAB host cell.

18. Distillers wet grains (DWG), distillers dried grains (DDG) or a syrup obtainable or obtained by the process of claim 15 and comprising a component of the recombinant LAB host cell.

19. Distillers wet grains with solubles (DWGS), dried solubles (DS) Distillers dried grains and solubles (DDGS) obtainable or obtained by the process of claim 16 and comprising a component of the recombinant LAB host cell.
